(19) United States
(12) Patent Application Publication
(10) Pub. No.: US 2017/0281914 A1
(43) Pub. Date: Oct. 5, 2017

(54) LOCALIZED TREATMENT OF TISSUES THROUGH TRANSCATHETER DELIVERY OF ACTIVE AGENTS

(71) Applicant: Surmodics, Inc., Eden Prairie, MN (US)

(72) Inventor: Joram Slager, St. Louis Park, MN (US)

(21) Appl. No.: 15/470,362

(22) Filed: Mar. 27, 2017

Related U.S. Application Data
(60) Provisional application No. 62/316,161, filed on Mar. 31, 2016.

(51) Int. Cl. A61M 25/10; A61M 25/00; A61K 31/337

(52) U.S. Cl. CPC A61M 25/10; A61K 31/337; A61M 25/0026; A61M 25/007; A61M 2025/1052

(57) ABSTRACT

Embodiments herein include catheters and methods for the localized treatment of tissues through transcatheter delivery of active agents. In an embodiment, a method herein can include inserting a catheter into the lumen of a blood vessel. The catheter can include an inflatable balloon, a first lumen within the shaft for delivering a fluid to inflate the balloon, an active agent delivery port, and a second lumen disposed within the shaft for delivering the active agent composition through the shaft to the active agent delivery port. The method can include inflating the balloon to at least partially occlude the flow of blood through the blood vessel. The method can include ejecting the active agent composition from the active agent delivery port into the blood vessel. In an embodiment, a catheter for treating a localized region of the body with an active agent composition is included. Other embodiments are also included herein.

This application claims the benefit of U.S. Provisional Application No. 62/316,161, filed Mar. 31, 2016, the contents of which are herein incorporated by reference.

### FIELD

Embodiments herein relate to catheters and methods for the localized treatment of tissues through transcatheter delivery of active agents.

### BACKGROUND

The efficacy of some active agents can depend on therapeutic titers reaching the appropriate anatomical location or compartment after administration. The phrase “route of administration” refers to the path by which an active agent is brought into contact with the body and is determined primarily by the properties of the active agent and by the therapeutic objectives. The route of intracorporeal administration that is chosen for a particular active agent may have a profound effect upon the speed and efficiency of the active agent upon administration.

In general, routes of administration can be classified by whether the effect is local or systemic. For local delivery, an active agent is typically applied directly to the tissue or organ for which treatment is sought. The effect of local delivery is limited primarily to the tissue or organ to which the active agent is applied. In contrast, an active agent administered systemically enters the blood or lymphatic supply and may have effects some distance from the site of administration.

### SUMMARY

Embodiments herein include catheters and methods for the localized treatment of tissues through transcatheter delivery of active agents. In an embodiment, a method of treating a localized region of the body with an active agent composition is included. The method can include inserting a catheter into a blood vessel of a patient. The catheter can include a shaft including a proximal end and a distal end. The catheter can further include an inflatable balloon, in fluid communication with a first lumen disposed within the shaft for delivering a fluid to inflate the balloon, an active agent delivery port, and a second lumen disposed within the shaft for delivering the active agent composition through the shaft to the active agent delivery port. The method can further include inflating the balloon to at least partially occlude the flow of blood through the blood vessel of the patient. The method can further include ejecting the active agent composition out of the active agent delivery port into the blood vessel. The active agent composition can include a cationic delivery agent, a therapeutically effective amount of the hydrophobic active agent, and a pharmaceutically acceptable carrier. The method can further include deflating the balloon to restore the flow of blood through the blood vessel and withdrawing the catheter from the blood vessel of the patient.

In an embodiment, a catheter for treating a localized region of the body with an active agent composition is included. The catheter can include a shaft including a proximal end and a distal end, an inflatable balloon, a first lumen disposed within the shaft for delivering a gas to inflate the balloon, an active agent delivery port, and a second lumen disposed within the shaft in fluid communication with the active agent delivery port. The catheter can further include an active agent composition disposed within the second lumen. The active agent composition can include a cationic delivery agent, a therapeutically effective amount of the hydrophobic active agent, and a pharmaceutically acceptable carrier.

In an embodiment, a kit for treating a localized region of the body with an active agent composition is included. The kit can include a catheter including a shaft including a proximal end and a distal end, an inflatable balloon, a first lumen disposed within the shaft for delivering a gas to inflate the balloon, an active agent delivery port, and a second lumen disposed within the shaft in fluid communication with the active agent delivery port. The kit can further include an active agent composition disposed within the second channel. The active agent composition can include a cationic delivery agent, a therapeutically effective amount of the hydrophobic active agent and a pharmaceutically acceptable carrier.

In an embodiment, a method of treating a localized region of the body with an active agent composition is included. The method includes inserting a catheter into the lumen of a blood vessel of a patient, the catheter including a shaft including a proximal end and a distal end, the catheter further including an inflatable balloon, a first lumen disposed within the shaft for delivering a fluid to inflate the balloon, an active agent delivery port, and a second lumen disposed within the shaft for delivering the active agent composition through the shaft to the active agent delivery port. The method can include inflating the balloon to at least partially occlude the flow of blood through the blood vessel of the patient. The method can include ejecting the active agent composition out of the active agent delivery port into the blood vessel. The active agent composition can include (a) a bioactive agent-containing particulate including: (i) a core particle including bioactive agent and a first biocompatible polymer; and a layer around the core particle including a second biocompatible polymer and that comprises negatively charged groups, wherein the second biocompatible polymer is more water soluble than the first biocompatible polymer; or

(ii) a particle including bioactive agent and a first biocompatible polymer that is water insoluble, wherein the polymer is chemically modified at the particle surface to provide negatively charged groups; and b) a cationic agent associated with the negatively charged groups of the particle. The method can also include deflating the balloon to restore the flow of blood through the blood vessel and withdrawing the catheter from the lumen of the blood vessel of the patient.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following drawings, in which:

FIG. 1 is a flowchart illustrating operations of a method in accordance with various embodiments herein.

FIG. 2 is a schematic diagram of a catheter in accordance with various embodiments herein.

FIG. 3 is a cross-sectional view of the catheter of FIG. 2 as taken along line 3-3′.

FIG. 4 shows paclitaxel concentration in various organs.

While embodiments are susceptible to various modifications and alternative forms, specifics thereof have been shown by way of example and drawings, and will be described in detail. It should be understood, however, that the scope herein is not limited to the particular embodiments described. On the contrary, the intention is to cover modifications, equivalents, and alternatives falling within the spirit and scope herein.

### DETAILED DESCRIPTION

The embodiments described herein are not intended to be exhaustive or to limit the invention to the precise forms disclosed in the following detailed description. Rather, the embodiments are chosen and described so that others skilled in the art can appreciate and understand the principles and practices.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

The flow of blood in many blood vessels can have relatively high velocities, such that components, including active agents, put into the blood vessels will quickly move away from the site at which they are first introduced into a blood vessel. This can be useful for active agents that are designed for systemic administration. However, this can be problematic for active agents designed to treat localized areas of the body because they can be quickly washed away into other sites within the body. In various embodiments herein, a balloon on a catheter is inflated so as to temporarily stop or slow the flow of blood and an active agent composition is ejected from the catheter, allowing the active agent to be used to treat a localized area of the body without being quickly washed away as would otherwise happen based on the flow of blood.

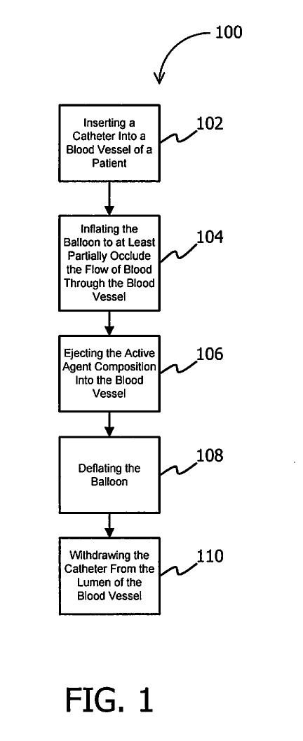

In an embodiment, a method of treating a localized region of the body with an active agent composition is included. Referring now to FIG. 1, a flowchart of an exemplary method 100 is shown. The method can include an operation of inserting 102 a catheter into the lumen of a blood vessel of a patient. The catheter can be inserted according to standard protocols used for inserting catheters into blood vessels.

It will be appreciated that the blood vessel can be a vein or an artery. Veins can include, but are not limited to, jugular, subclavian, cephalic, brachial, basilica, iliac, saphenous, and branches and sub-branches of all of these. Arteries can include, but are not limited to, coronary arteries, brachiocephalic arteries, carotid arteries, subclavian arteries, femoral arteries, popliteal arteries, tibial arteries, the celiac artery, the left gastric artery, the common hepatic artery, the splenic artery, the superior mesenteric artery, the inferior pancreaticoduodenal artery, the intestinal arteries, the ileocolic artery, the right colic artery, the middle colic artery, the renal artery, the inferior mesenteric artery, the left colic artery, the sigmoid branches, the superior rectal artery, and branches and sub-branches of all of these. In some embodiments the artery can be a renal artery or a hepatic artery. The localized region being treated can correspond to an area of the body and/or a specific organ or sub-portion of an organ. Specific organs can include, but are not limited to, the heart, the liver, the kidneys, the pancreas, the spleen, the lungs, or any tumor-containing organ.

The method can further include an operation of inflating 104 the balloon to at least partially occlude the flow of blood through a targeted organ or the blood vessel of the patient. The balloon can be inflated by delivering a fluid (such as a gas or a liquid) through a lumen disposed within catheter the shaft for delivering a fluid to inflate the balloon. In some embodiments, the balloon is completely filled with a fluid. In other embodiments, the balloon is partially filled with a fluid. In some embodiments, inflating the balloon completely occludes the flow of blood in the vessel. In some embodiments inflating the balloon causes the peak systolic velocity of blood flow through the artery to be reduced by more than 75%. In some embodiments inflating the balloon causes the peak systolic velocity of blood flow through the artery to be reduced to less than 2 cm/sec.

Other exemplary methods for occluding blood flow to deliver formulations of the present disclosure can include, but are not limited to, mechanically occlusive devices such as those described in US Pat. Publication No. 2015/0343181 (to Bradway et al.). The occluding member can have a radially-outwardly expanded configuration and a contracted configuration. A first biasing member can be disposed within the occluding member having a first end adjoined to the proximal end of the device, and a second biasing member can be disposed within the occluding member having a first end adjoined to the distal end of the device. The second biasing member can be configured to releasably engage the first biasing member.

It will be appreciated that the method can also include a step of moving the catheter within the lumen of the blood vessel to position the balloon in a desired location prior the step of inflating the balloon. Positioning can be performed and/or assisted using various imaging techniques. In some embodiments, the catheter can include one or more radiopaque regions in order to assist in imaging of the position of the catheter.

The method can further include an operation of ejecting 106 the active agent composition out of the active agent delivery port into the blood vessel. The active agent active agent composition can be transferred from within the catheter and into the vessel by passing through the active agent delivery port. The active agent composition can flow from an area of higher pressure, such as inside the catheter, to a region of lower pressure, such as within the lumen of the blood vessel. This can be achieved simply by pushing the active agent composition out of the catheter as would happen when the composition is being continuously injected into the catheter through a port on or near the manifold on the proximal end catheter. However, other techniques for ejecting the active agent are also contemplated herein.

The method can further include an operation of deflating 108 the balloon to restore the flow of blood through the blood vessel. In some embodiments, the method can include a waiting period between ejection 106 and deflating 108. In some cases, the waiting period can be from 10 seconds to 4 minutes. In some cases the waiting period can be from 30 seconds to 2 minutes. In some embodiments, after balloon inflation, a valve is closed or another method of maintaining the fluid within the balloon is used. The balloon can be deflated by withdrawing a fluid from the balloon, such as by opening a valve or withdrawing the fluid using a syringe or other instrument. The method can further include an operation of withdrawing 110 the catheter from the lumen of the blood vessel of the patient.

Catheters herein can include a shaft including a proximal end and a distal end. The catheter can further include an inflatable balloon, a first lumen disposed within the shaft for delivering a fluid to inflate the balloon, an active agent delivery port (or infusion port), and a second lumen disposed within the shaft for delivering the active agent composition through the shaft to the active agent delivery port.

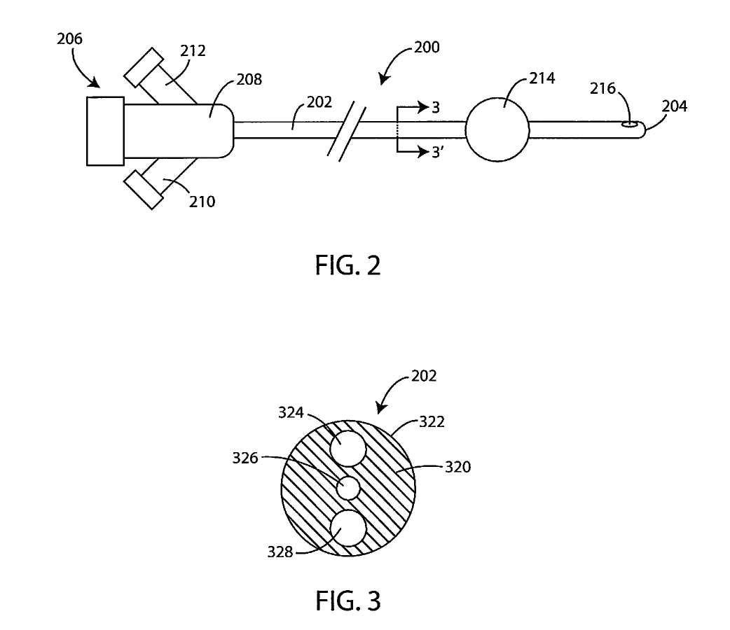

Referring now to FIG. 2, a diagram of an exemplary catheter 200 (not to scale) is shown for purposes of illustration, though it will be appreciated that many different specific catheters design are contemplated herein. The catheter 200 can include a shaft 202 having a distal end 204 and a proximal end 206. The shaft can be formed of various materials including, but not limited to, silicones, polyvinyl chloride (PVC), latex rubber, polyamides, polyurethane, polyethylene terephthalate, PEBAX, and the like. A manifold 208 can be disposed on the proximal end 206 of the catheter 200. Ports 210 and 212 can be disposed on or near the manifold 208. By way of example, one port can be used for the injection of a fluid to inflate a balloon and the other port can be used to inject an active agent composition. It will be appreciated, however, that while this figure only shows two ports, there can be greater or fewer than two. A guidewire, not shown, can be threaded in from the back of the manifold 208. A balloon 214 can be disposed on the catheter shaft 202. The balloon 214 can be formed of various materials including, but not limited to, natural or synthetic elastomeric polymers, expandable materials, and the like. In many cases, the balloon is compliant. However, in some embodiments the balloon can be non-compliant. An active agent delivery port 216, which can take the form of an aperture in the surface of the catheter shaft 202 in some embodiments, can be disposed on the shaft 202 at a position closer towards the distal end 204 of the catheter shaft 202 than the balloon 214. In some embodiments, the active agent delivery portion 216 can be disposed on the side of the shaft 202, such as shown in FIG. 2. However, in other embodiments, the active agent delivery port 216 can be disposed on the tip of the distal end 204 of the catheter shaft 202.

Referring now to FIG. 3, a cross-sectional view of a catheter shaft is shown as taken along line 3-3′ of FIG. 2. The catheter shaft 202 includes a body member 320 and has an outer surface 322. Various lumens (or channels) can be disposed within the body member 320 of the catheter shaft 202. In specific, by way of example, the body member 320 can include a balloon fluid lumen 324, a guide wire lumen 326, and an active agent lumen 328. Various other lumens can also be included. Additional aspects of catheter constructions are disclosed in U.S. Pat. Nos. 6,997,898; 8,088,103; 8,162,879; and 8,613,721, the content of which related to catheters is herein incorporated by reference.

It will be appreciated that while the catheter shown in FIG. 2 illustrates a single balloon, embodiments herein can also include catheters with multiple balloons. For example, catheters in accordance with embodiments herein can include two, three or more balloons. In some embodiments, the balloons are all disposed on the catheter shaft on the same side of the active agent delivery port (e.g., toward the proximal or distal end of the catheter shaft from the active agent delivery port). However, in some embodiments, at least one balloon can be on an opposite side of the active agent delivery port (e.g., at least one balloon can be between the active agent delivery port and the proximal end of the catheter shaft and at least one balloon can be between the active agent delivery port and the distal end of the catheter shaft).

The active agent composition delivered through the catheter can include a cationic delivery agent, a therapeutically effective amount of the hydrophobic active agent, and a pharmaceutically acceptable carrier. Aspects of these components will now be discussed in greater detail, it being appreciated that the scope of embodiments herein are not limited to these specific examples.

### Hydrophobic Active Agents

In various embodiments, the active agent composition includes one or more hydrophobic active agents. In general, the term “hydrophobic active agent” refers to an active agent having solubility in water of less than about 100 μg/mL at 25° C. and neutral pH, less than about 10 μg/mL at 25° C. and neutral pH, or less than about 5 μg/ml at 25° C. and neutral pH. In various embodiments, the hydrophobic active agent is crystalline. In general, the term “crystalline” refers to a thermodynamically stable solid form of an active agent having “long range molecular order” in which the molecules are packed in a regularly ordered, repeating pattern. In another embodiment, the hydrophobic active agent is amorphous. The term “amorphous” refers to a solid form of an active agent in which the molecules do not have “long range molecular order”, but rather are randomly arranged or retain only a “short range molecular order” typical of liquids.

The amount of hydrophobic active agent included in the active agent composition can vary depending upon many factors including the desired therapeutic outcome. However, compositions herein generally include at least about 1 mg/ml, 2 mg/ml, 3 mg/ml, 4 mg/ml, 5 mg/ml, or 10 mg/ml, 15 mg/ml, 20 mg/ml, or 25 mg/ml or up to about 25 mg/ml, 50 mg/ml, 75 mg/ml, 100 mg/ml, 125 mg/ml or 150 mg/ml hydrophobic active agent.

It will be appreciated that hydrophobic active agents can include agents having many different types of activities. In some embodiments, hydrophobic active agents can include, but are not limited to, antiproliferatives such as paclitaxel and analogues thereof, sirolimus (rapamycin), everolimus, biolimus A9, zotarolimus, tacrolimus, pimecrolimus and other sirolimus derivatives, and mixtures thereof; analgesics and anti-inflammatory agents such as aloxiprin, auranofin, azapropazone, benorylate, diflunisal, etodolac, fenbufen, fenoprofen calcium, flurbiprofen, ibuprofen, indomethacin, ketoprofen, meclofenamic acid, mefenamic acid, nabumetone, naproxen, oxyphenbutazone, phenylbutazone, piroxicam, sulindac; anti-arrhythmic agents such as amiodarone HCl, disopyramide, flecainide acetate, quinidine sulphate; anti-bacterial agents such as benethamine penicillin, cinoxacin, ciprofloxacin HCl, clarithromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, trimethoprim; anti-coagulants such as dicoumarol, dipyridamole, nicoumalone, phenindione; anti-hypertensive agents such as amlodipine, benidipine, darodipine, dilitazem HCl, diazoxide, felodipine, guanabenz acetate, isradipine, minoxidil, nicardipine HCl, nifedipine, nimodipine, phenoxybenzamine HCl, prazosin HCL, reserpine, terazosin HCL; anti-muscarinic agents: atropine, benzhexol HCl, biperiden, ethopropazine HCl, hyoscyamine, mepenzolate bromide, oxyphencylcimine HCl, tropicamide; anti-neoplastic agents and immunosuppressants such as aminoglutethimide, amsacrine, azathioprine, busulphan, chlorambucil, cyclosporin, dacarbazine, estramustine, etoposide, lomustine, melphalan, mercaptopurine, methotrexate, mitomycin, mitotane, mitozantrone, procarbazine HCl, tamoxifen citrate, testolactone; beta-blockers such as acebutolol, alprenolol, atenolol, labetalol, metoprolol, nadolol, oxprenolol, pindolol, propranolol; cardiac inotropic agents such as amrinone, digitoxin, digoxin, enoximone, lanatoside C, medigoxin; corticosteroids such as beclomethasone, betamethasone, budesonide, cortisone acetate, desoxymethasone, dexamethasone, fludrocortisone acetate, flunisolide, flucortolone, fluticasone propionate, hydrocortisone, methylprednisolone, prednisolone, prednisone, triamcinolone; lipid regulating agents such as bezafibrate, clofibrate, fenofibrate, gemfibrozil, probucol; nitrates and other anti-anginal agents such as amyl nitrate, glyceryl trinitrate, isosorbide dinitrate, isosorbide mononitrate, pentaerythritol tetranitrate.

Other hydrophobic active agents include, but are not limited to, active agents for treatment of hypertension (HTN), such as guanethidine.

In a particular embodiment, the hydrophobic active agent includes paclitaxel, sirolimus (rapamycin), everolimus, biolimus A9, zotarolimus, tacrolimus, and pimecrolimus and mixtures thereof.

In some embodiments, the hydrophobic active agent includes chemotherapeutics, exemplified by the family of fluorouracils (e.g. 4-FU and 5-FU) and carmustine (BCNU 1,3-bis (2-chloroethyl)-1-nitrosourea) and temozolomide.

In various embodiments, the hydrophobic active agent is combined with a cationic delivery agent in solution. In various embodiments, the hydrophobic active agent is combined with a cationic delivery agent to form a suspension. In another embodiment, solid hydrophobic active agent, amorphous or crystalline, is combined with pure or neat cationic delivery agent, amorphous or crystalline, to form a mixture. In other embodiments, the hydrophobic active agents is conjugated to a cationic delivery agent. The conjugation can include a hydrophobic active agent covalently bonded to the cationic delivery agent. In some embodiments wherein the hydrophobic agent is conjugated to the cationic delivery agent a linking agent can be used to attach the hydrophobic agent to the cationic delivery agent. Suitable linking agents include, but are not limited to, polyethylene glycol, polyethylene oxide and polypeptides of naturally-occurring and non-naturally occurring amino acids. In some embodiments, linking agents can be biodegradable or cleavable in vivo to assist in release of the hydrophobic active agents. Exemplary linking agents can further include alkane or aromatic compounds with heteroatom-substitutions such as N, S, Si, Se or O.

In various embodiments the active agent can be part of a microparticle. The term “microparticle” as used herein shall refer to non-dissolved particulate matter. In some embodiments, microparticles can include a polymer. In some embodiments, the microparticle can include a polymer that is distinct from the other polymers that may be used. Including a polymer within the microparticle can offer the advantage of providing additional control over the elution rate of the active agent. In some embodiments, including a polymer within the microparticle can also offer the advantage of increased protection of active agent activity.

Both degradable and non-degradable polymers can be used in embodiments of the invention. The use of degradable polymers in the elution control coating can offer the advantage of controlling elution rate of an active agent without depending solely on the process of the active agent diffusing through the matrix itself. Rather, as the matrix erodes (through bulk or surface erosion) the active agent is released into the local environment of the elution control coating. The term “degradable” as used herein with reference to polymers, shall refer to those natural or synthetic polymers that break down under physiological conditions into constituent components over a period of time. By way of example, many degradable polymers include hydrolytically unstable linkages in the polymeric backbone. The cleavage of these unstable linkages leads to degradation of the polymer. The terms “erodible”, “bioerodible”, “biodegradable” and “non-durable” shall be used herein interchangeably with the term “degradable”.

The polymer used with the microparticle can be degradable or non-degradable. A specific polymer can be selected based on various factors including compatibility with the active agent, whether or not the polymer is degradable, speed and habit of erosion (bulk or surface), and compatibility or incompatibility with solvents used to apply the coating.

In an embodiment, the microparticle includes a degradable polymer. Elution of an active agent from a particle including a degradable polymer can be from diffusion of the active agent through the degradable polymer itself or through the erosion (bulk or surface erosion) of the degradable polymer. Degradable polymers can include those described in more detail below.

Microparticles used with embodiments of the invention may be configured to provide a desired active agent elution rate. The rate of active agent elution from a microparticle will depend on various factors including the size of the microparticle, the presence or absence of other components in the microparticle such as a polymer, an additive, or a solvent, the erosion characteristics of the material in the microparticle, the structural features of the microparticle including porosity, overcoats and the like. By way of example, microparticles with a larger diameter may elute an active agent more slowly than microparticles of a smaller diameter. In addition, microparticles with too large of a diameter may result in a coating with a rough surface and may clog coating equipment. In some embodiments, microparticles used with embodiments of the invention have an average size distribution in the range between about 10 nm to about 100 μm as measured by SEM analysis. In an embodiment, microparticles are equal to or less than about 5 μm.

In some embodiments, the microparticles used are substantially monodisperse. In other embodiments, the microparticles used are polydisperse. In some applications, the use of monodisperse microparticles is advantageous because elution rates from monodisperse microparticles can be more consistent than release rates from otherwise similar polydisperse microparticles.

Microparticles having a characteristic elution rate can be combined with other microparticles having the same or a different characteristic elution rate. By combining particles with different characteristic release rates, the overall release rate of an active agent from the particles and from the matrix that the particles are dispersed in can be manipulated as desired. For example, microparticles having a relatively fast elution rate can be combined in a coating with microparticles having a relatively slow elution rate to produce a composition elution profile that is desirable.

In some embodiments, various additives can be used to enhance the stability of the suspension of microparticles. Additives may include various components such as surfactants, stabilizers, etc. In some embodiments, a polymer is used to enhance the stability of the suspension of microparticles. By way of example, addition of a (poly(butyleneterephthalate-co-ethylene glycol) copolymer can enhance the stability of the suspension.

In some embodiments, particulates herein are formed by preparing a drug-containing particle (e.g., a core particle) along with biocompatible polymer that has negatively charged groups or that can be treated to provide negatively charged groups, and then associating a cationic agent with the negatively charged groups of the particulate to provide the particulate with a positive zeta-potential. Drug-containing particulates that can be used in the embodiments as described herein are detailed in the provisional application Ser. 62/315,917 entitled “Drug-Containing Particulate Composition with Cationic Agent, Associated Medical Devices, and Methods for Treatment” to Slager; filed Mar. 31, 2016, the entire application of which is incorporated herein by reference.

In one aspect (e.g., a first particle aspect of the disclosure), the bioactive agent particulates include a core particle having a bioactive agent and a first biocompatible polymer. There is a layer around the core particle that includes a second biocompatible polymer which includes negatively charged groups. The second biocompatible polymer is chosen to be more water soluble than the first biocompatible polymer. A cationic agent is associated with the negatively charged groups of particulate in a manner that provides the particulate with a positive zeta-potential. “Biocompatible” polymers are those that do not provoke any significant adverse effects when introduced into the body.

In a mode of practice, the particles of the first particle aspect of the disclosure can be prepared by first preparing a first composition that includes the bioactive agent and first biocompatible polymer. Next, the first composition is contacted with a composition that includes the second biocompatible polymer having negatively charged groups. This forms an intermediate particle with negatively charged groups on the outer surface, and a core particle including the bioactive agent and first biocompatible polymer. Next, the intermediate particle is contacted with a composition that include cationic agent so that the resulting particulate has a positive zeta-potential.

The first biocompatible polymer can be an organic solvent-soluble degradable polymer. Examples of degradable polymers can include those with hydrolytically unstable linkages in the polymeric backbone. The degradable polymers can exhibit bulk erosion or surface erosion characteristics. Synthetic degradable polymers can include: degradable polyesters (such as poly(glycolic acid), poly(lactic acid), poly(lactic-co-glycolic acid), poly(dioxanone), polylactones (e.g., poly(caprolactone)), poly(3-hydroxybutyrate), poly(3-hydroxyvalerate), poly(valerolactone), poly(tartronic acid), poly(β-malonic acid), poly(propylene fumarate)); degradable polyesteramides; degradable polyanhydrides (such as poly(sebacic acid), poly(1,6-bis(carboxyphenoxy)hexane, poly(1,3-bis(carboxyphenoxy)propane); degradable polycarbonates (such as tyrosine-based polycarbonates); degradable polyiminocarbonates; degradable polyarylates (such as tyrosine-based polyarylates); degradable polyorthoesters; degradable polyurethanes; degradable polyphosphazenes; and degradable polyhydroxyalkanoates; and copolymers thereof.

In some aspects the second polymer is a biodegradable block copolymer including hydrophilic and hydrophobic blocks. The linkages between the blocks can be biodegradable or biostable, and the hydrophilic and hydrophobic blocks can be either or both biodegradable or biostable, with at least one portion of the copolymer being biodegradable.

In some aspects the hydrophobic blocks include a biodegradable polymeric segment selected from polycaprolactone (PCL), polyvalerolactone (PVL), poly(lactide-co-glycolide) (PLGA), polylactic acid (PLA), polybutyrolactone (PBL), polyglycolide, polypropiolactone (PPL), and polytrimethylene carbonate.

Exemplary hydrophilic blocks can be selected from polymer segments formed from monomers such as ethylene glycol, ethylene oxide, vinyl alcohol, propylene oxide, vinyl pyrrolidone, hydroxy ethyl methacrylate, and hydroxy ethyl acrylate.

Exemplary hydrophilic blocks include (PEO), polyvinyl alcohol (PVA), poly(vinyl pyrrolidone) (PVP), polyacrylamide, poly(hydroxy alkyl methacrylate), poly(hydroxy ethyl methacrylate), hydrophilic polyurethane, HYPAN, oriented HYPAN, poly(hydroxy ethyl acrylate), poly(ethyloxazoline), and polyamines (e.g., Jeffamine™).

In some aspects the second polymer comprises a polyalkoxyalkane block. Representative examples of polyalkoxyalkane blocks include poly(ethylene glycol), tetraethylene glycol, triethylene glycol, trimethylolpropane ethoxylate, and pentaeerythritol etholxylate blocks.

Exemplary hydrophilic blocks have a molecular weight of about 100 Da to about 5000 Da, or about 250 Da to about 3500.

In some aspects, the degradable polymers include at least two hydrolysable segments derived from pre-polymers A and B, which segments are linked by a multi-functional chain-extender and are chosen from the pre-polymers A and B, and triblock copolymers ABA and BAB, wherein the multi-block copolymer is amorphous and has one or more glass transition temperatures (Tg) of at most 37° C. (Tg) at physiological (body) conditions. The pre-polymers A and B can be a hydrolysable polyester, polyetherester, polycarbonate, polyestercarbonate, polyanhydride or copolymers thereof, derived from cyclic monomers such as lactide (L,D or L/D), glycolide, ε-caprolactone, δ-valerolactone, trimethylene carbonate, tetramethylene carbonate, 1,5-dioxepane-2-one, 1,4-dioxane-2-one (para-dioxanone) or cyclic anhydrides (oxepane-2,7-dione). The composition of the pre-polymers may be chosen in such a way that the maximum glass transition temperature of the resulting copolymer is below 37° C. at body conditions. To fulfill the requirement of a Tg below 37° C., some of the above-mentioned monomers or combinations of monomers may be more preferred than others. This may by itself lower the Tg, or the pre-polymer is modified with a polyethylene glycol with sufficient molecular weight to lower the glass transition temperature of the copolymer. The degradable multi-block copolymers can include hydrolysable sequences being amorphous and the segments may be linked by a multifunctional chain-extender, the segments having different physical and degradation characteristics. For example, a multi-block co-polyester consisting of a glycolide-ε-caprolactone segment and a lactide-glycolide segment can be composed of two different polyester pre-polymers. By controlling the segment monomer composition, segment ratio and length, a variety of polymers with properties that can easily be tuned can be obtained. Such degradable multi-block copolymers can specifically include those described in U.S. Publ. App. No. 2007/0155906, the content of which is herein incorporated by reference in its entirety.

Specific examples of these types of degradable copolymers include poly(ether ester) multiblock copolymers based on poly(ethylene glycol) (PEG) and poly(butylene terephthalate) (PBT) that can be described by the following general structure:

[—(OCH₂CH₂)ₙ—O—C(O)—C₆H₄—C(O)—]ₓ[—O—(CH₂)₄—O—C(O)—C₆H₄—C(O)—]_{y},

where —C₆H₄— designates the divalent aromatic ring residue from each esterified molecule of terephthalic acid, n represents the number of ethylene oxide units in each hydrophilic PEG block, x represents the number of hydrophilic blocks in the copolymer, and y represents the number of hydrophobic blocks in the copolymer. In embodiments, n can be selected such that the molecular weight of the PEG block is between about 300 and about 4000. X and y can be selected so that the multiblock copolymer contains from about 55% up to about 80% PEG by weight. The properties of copolymer can be changed by varying the values of n, x and y in the copolymer structure. An exemplary copolymer of this class is PEG₁₀₀₀-45PBT-55 which is a copolymer of a poly(butyleneterephthalate-co-ethylene glycol) copolymer with 45 wt. % polyethylene glycol having an average molecular weight of 1000 kD and 55 wt. % butyleneterephthalate. PEG₁₀₀₀-45PBT-55 is commercially available from OctoPlus (Leiden, Netherlands) under the product name PolyActive™.

Another example of these types of degradable copolymers include poly(ether ester) multiblock copolymers based on poly(ethylene glycol) (PEG) and one or more of glycolide, lactide, and/or caprolactone monomers or polymer segments. As specific example is 20GAPEGCL-80GALA, which is a block copolymer of 20 wt. % glycolide-polyethylene glycol-caprolactone “GAPEGCL” and 80 wt, % glycolide-lactide “GALA”.

An initial step in the process of preparing the first particle aspect of the disclosure involves preparing a liquid composition including the bioactive agent and the first degradable biocompatible polymer. A solvent or solvent mixture can be chosen to dissolve both the bioactive agent and the first degradable biocompatible polymer.

Exemplary solvents or dispersant include, but are not limited to, aromatic hydrocarbons, such as benzene, xylene (e.g., ortho-xylene, para-xylene, or meta-xylene) and toluene; C1-C4 alcohols such as methanol, ethanol (EtOH), isopropanol (IPA), n-butanol, isobutyl alcohol and t-butyl alcohol; halogenated organic solvents such as dicholoroethane (DCE), dichloromethane (DCM), chloroform, and ethyl trifluroacetate (ETFA); ketones such as methyl isobutyl ketone (MIBK), 3-pentanone (diethyl ketone) acetone, 2-butanone (MEK); acetonitrile (ACN); ethers such as isopropyl ether (IPE) and tetrahydrofuran (THF); aliphatic hydrocarbons such as hexane, heptane, or the like; and esters such as ethyl acetate and butyl acetate.

An “acid group-containing polymer” refers to polymer that has acid groups presented on the polymer chain, and which can provide negatively charged groups to the particulate. Acidic groups include, for example, sulfonic acids, carboxylic acids, phosphonic acids, and the like. Exemplary salts of such groups include, for example, sulfonate, carboxylate, and phosphate salts. Exemplary counter ions include alkali, alkaline earths metals, ammonium, protonated amines, and the like. If one or more counter ions are used, the acid groups of the acid group-containing polymer can be partially neutralized.

Exemplary carboxylic acid-group containing monomers that can be used to prepare the acid group-containing polymer, include, but are not limited to acrylic acid, methacrylic acid, itaconic acid, monomethyl itaconic acid, maleic anhydride, fumaric acid, and crotonic acid, and salts thereof. Exemplary sulfonic acid-group containing monomers that can be used to prepare the acid group-containing polymer, include, but are not limited to acrylamido-2-methylpropanesulfonic acid (AMPS), 2-(meth)acrylamido-2-methylpropane sulfonic acid, vinyl sulfonic acid, 2-sulfoethyl methacrylate, and salts thereof. Other exemplary carboxylic acid-containing monomers that can be used to prepare the acid group-containing copolymers include styrene and maleic anhydride copolymerized to produce styrene-maleic anhydride copolymer (PSMA).

### Cationic Delivery Agents

In various embodiments, the active agent composition includes a hydrophobic active agent and cationic delivery agent. While not wishing to be bound by theory, it is believed that the charge provided by the cationic delivery agents results in the composition being electrostatically attracted to negative charges and/or polar groups associated with the lipid bilayer present on or in a tissues or organs of a patient or charged/polar groups associated with the extracellular matrix (e.g., collagen, fibronectin, laminin, etc.). Consequently, combining an active agent, particularly a hydrophobic active agent with a cationic delivery agent in a composition for local administration can help retain the hydrophobic active agent near the site of administration. It is also thought that the cationic delivery agent may increase tissue permeability, thereby enhancing uptake of the active agent by the target tissue and/or organ.

In general, the upper limit for the amount of cationic delivery agent that is included in the active agent composition is guided by the toxicity limit for the given cationic delivery agent or the solubility of the cationic delivery agent in the aqueous carrier used in the composition. However, in one embodiment, the ratio of cationic delivery agent:hydrophobic active agent can be up to 1:1. The lower limit for the amount of cationic delivery agent that is included in the composition is guided by the efficacy of the composition. In general, the inventors have found that a ratio of cationic delivery agent:hydrophobic active agent of 1:50 and less has limited efficacy. Consequently, the composition generally has a ratio of cationic delivery agent: hydrophobic active agent of at least 1:25. In various embodiments, the ratio of cationic delivery agent:hydrophobic active agent is between about 1:1 and about 1:25. In another embodiment, the ratio of cationic delivery agent:hydrophobic active agent is at least about 1:2, 1:5 or 1:10 and up to about 1:10, 1:15, 1:20 or 1:25. In some embodiments, the composition herein includes at least about 0.1 mg/ml, 0.5 mg/ml, 1 mg/ml, 2 mg/ml, 3 mg/ml, 4 mg/ml, or 5 mg/ml and up to about 5 mg/ml, 10 mg/ml, 15 mg/ml, 20 mg/ml or 25 mg/ml cationic delivery agent.

Cationic delivery agents used in embodiments herein include compounds containing a portion having a positive charge in aqueous solution at neutral pH along with a portion that can exhibit affinity for hydrophobic surfaces (such as hydrophobic or amphiphilic properties) and can therefore interface with hydrophobic active agents. In some embodiments, cationic delivery agents used in embodiments herein can include those having the general formula X—Y, wherein X is a positively charged group in aqueous solution at neutral pH and Y is a moiety exhibiting hydrophobic properties. In some embodiments, the cationic delivery agent can include a hydrophilic head and a hydrophobic tail, along with one or more positively charged groups, typically in the area of the hydrophilic head.

Cationic delivery agents can specifically include cationic lipids and net neutral lipids that have a cationic group. Exemplary lipids can include, but are not limited to, 3β-[N—(N′,N′-dimethylaminoethane)-carbamoyl]cholesterol hydrochloride (DC-cholesterol); 1,2-dioleoyl-3-trimethylammonium-propane (DOTAP); dimethyldioctadecylammonium (DDAB); 1,2-dioleoyl-sn-glycero-3-ethylphosphocholine (EPC); 1,2-di-O-octadecenyl-3-trimethylammonium propane (DOTMA); 1,2-di-(9Z-octadecenoyl)-3-dimethylammonium-propane (DODAP); 1,2-dilinoleyloxy-3-dimethylaminopropane (DLinDMA) and derivatives thereof. Additional lipids can include, but are not limited to, 1,2-dioleoyl-sn-glycero-3-phosphoethanolamine (DOPE); cholesterol; 1,2-dioctadecanoyl-sn-glycero-3-phosphocholine (DSPC); 1,2-distearoyl-sn-glycero-3-phosphoethanolamine (DSPE).

Cationic delivery agents can specifically include cationic polymers. Cationic delivery agents can also include polycation-containing cyclodextrin, histones, protamines, cationized human serum albumin, aminopolysaccharides such as chitosan, peptides such as poly-L-lysine, poly-L-ornithine, and poly(4-hydroxy-L-proline ester, and polyamines such as polyethylenimine (PEI; available from Sigma Aldrich), polypropylenimine, polyamidoamine dendrimers (PAMAM; available from Sigma Aldrich), cationic polyoxazoline, polyvinylamine (PVAm), and poly(beta-aminoesters). Cationic delivery agents can also specifically include cationic lipidoids (as described by K. T. Love in the publication PNAS 107, 1864-1869 (2010)). Other exemplary cationic polymers include, but are not limited to, block copolymers such as PEG-PEI and PLGA-PEI copolymers.

In various embodiments, the cationic delivery agent includes polyethylenimine (PEI). PEI is a basic cationic aliphatic polymer which can be linear or branched. PEI herein can specifically include water soluble PEI. Linear PEI is a solid at room temperature and includes predominantly secondary amines and is soluble in water at relatively low molecular weights. Branched PEIs are liquid at room temperature and include primary, secondary and tertiary amino groups and is typically soluble in water. The ratio of primary:secondary:tertiary amino groups reflects the amount of branching, wherein the relative amount of secondary amino groups decreases as the amount of branching increases. In various embodiments, PEI includes primary:secondary:tertiary amino groups at a ratio of between about 1:3:1 and 1:1:1, or between about 1:2:1 and 1:1:1. In another embodiment, PEI includes primary:secondary:tertiary amino groups at a ratio of between about 1:2:1 and 1:1:1, 1:1.1:1, 1:1.2:1, 1:1.3:1, 1:1.4:1, 1:1.5:1, 1:1.6:1, 1:1.7:1, 1:1.8:1, or 1:1.9:1. In another embodiment, PEI is linear and includes predominantly secondary amines. In various embodiments, branched PEI includes no more than about 30%, 35%, 40%, 45%, 50%, 55%, 60%, 65%, 70%, or 75% secondary amine groups. In other embodiments, PEI includes one or more quaternary amine groups.

In one method, PEI is synthesized from monomers that include a three-membered ring in which two corners of the molecule have (—CH₂—) linkages and the third corner includes a secondary amine group (═NH). In the presence of a catalyst the three-membered ring is converted into a highly branched polymer with about 25% primary amine groups, 50% secondary amine groups, and 25% tertiary amine groups. The branched polymers can be copolymerized to produce PEI having a variety of molecular weights, from 2 kD up to 5000 kD. In various embodiments, PEI has a molecular weight of at least about 25 kD, 50 kD, 70 kD, 75 kD, 100 kD, 150 kD, 200 kD, 250 kD, 300 kD, 350 kD, 400 kD, 450 kD, 500 kD, 550 kD, 600 kD, 650 kD, 700 kD, 750 kD, 800 kD, 850 kD, 900 kD, 950 kD or 1000 kD and up to about 1000 kD, 1500 kD, 2000 kD, 2500 kD, 3000 kD, 3500 kD, 4000 kD, 4500 kD or 5000 kD. Methods for synthesizing linear PEI are also known.

In other embodiments of the present disclosure, cationic delivery agents having a positive charge in aqueous solutions at neutral pH include the following Compounds (A-I):

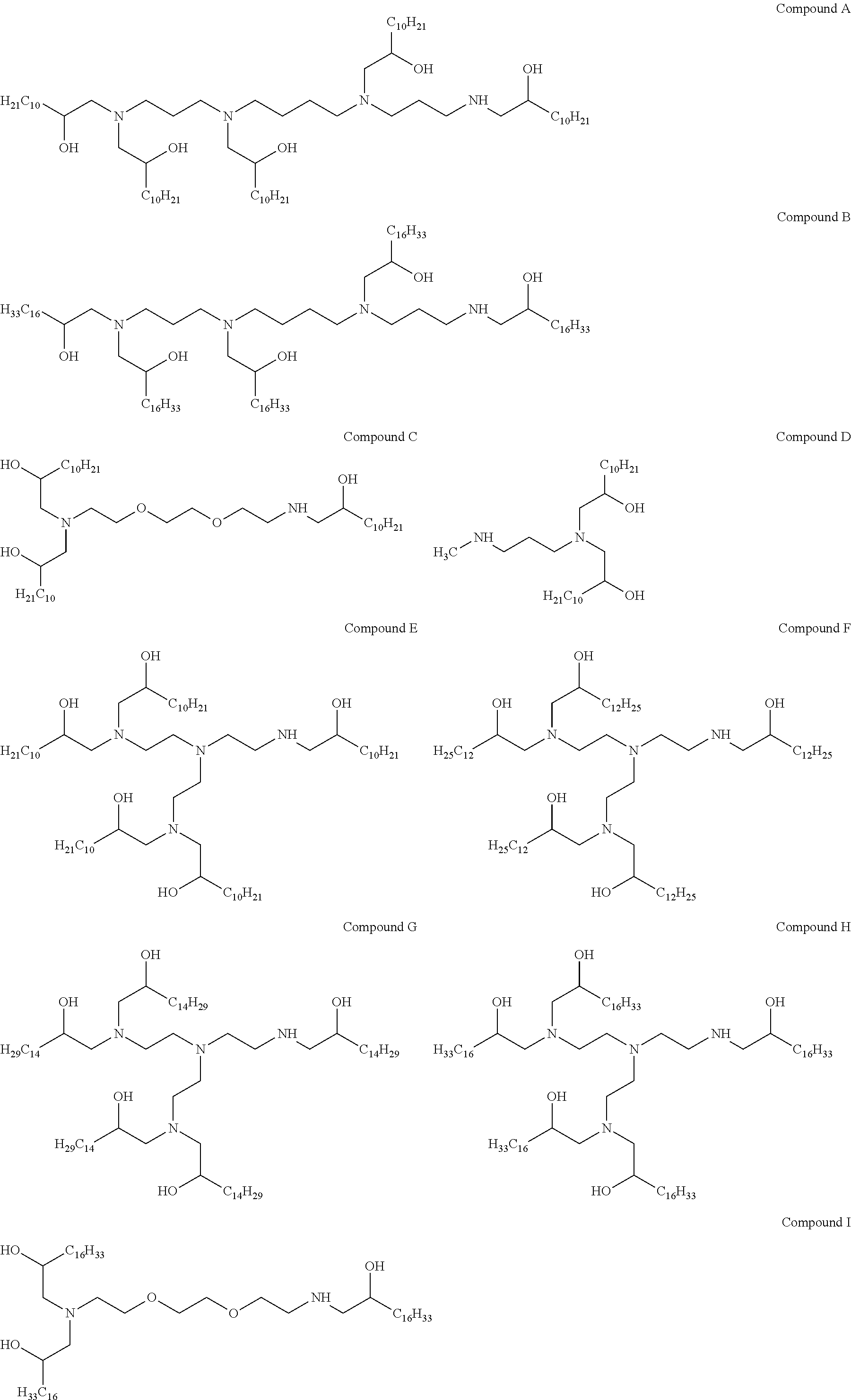

Additionally, other cationic delivery agents include structures of the general Formula I:

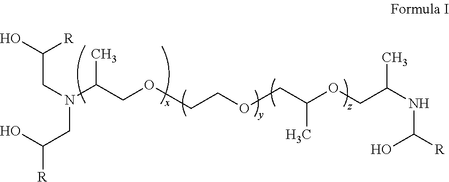

TABLE 1
Values for Variables x + z, y and R for Compounds J-R of Formula I.

| Compound | x + z | y | R |
|---|---|---|---|
| Compound J | 6 | 12.5 | C₁₂H₂₅ |
| Compound K | 1.2 | 2 | C₁₂H₂₅ |
| Compound L | 6 | 39 | C₁₂H₂₅ |
| Compound M | 6 | 12.5 | C₁₄H₂₉ |
| Compound N | 1.2 | 2 | C₁₄H₂₉ |
| Compound O | 6 | 39 | C₁₄H₂₉ |
| Compound P | 6 | 12.5 | C₁₆H₃₃ |
| Compound Q | 1.2 | 2 | C₁₆H₃₃ |
| Compound R | 6 | 39 | C₁₆H₃₃ |

Methods for making cationic delivery agents, such as those listed above, are described in more detail in U.S. patent application Ser. No. 13/469,844, entitled “DELIVERY OF COATED HYDROPHOBIC ACTIVE AGENT PARTICLES,” the disclosure of which is hereby incorporated by reference herein in its entirety. In general, cationic delivery agents, such as those listed above, can generally be prepared by the reaction of an appropriate hydrophobic epoxide (e.g. oleyl epoxide) with a multi-functional amine (e.g. propylene diamine). Details of the synthesis of related cationic delivery agents are described by K. T. Love in the publication PNAS 107, 1864-1869 (2010) and Ghonaim et al., Pharma Res 27, 17-29 (2010).

It will be appreciated that polyamide derivatives of PEI (PEI-amides) can also be applied as cationic delivery agents. PEI-amides can generally be prepared by reacting PEI with an acid or acid derivative such as an acid chloride or an ester to form various PEI-amides. For example, PEI can be reacted with methyl oleate to form PEI-amides.

In yet other embodiments cationic delivery agents can include moieties used to condense nucleic acids (for example lipids, peptides and other cationic polymers). In some instances these cationic delivery agents can be used to form lipoplexes and polyplexes.

### Additional Components

In other embodiments, the active agent compositions herein can include one or more additional components, such as a diluent, excipient, adjuvant, emulsifier, buffer, stabilizer, preservative, and the like. In various embodiments, the active agent composition includes one or more contrast agents, for example, an iodinated radiocontrast agent.

In another embodiment, the active agent compositions can include one or more agents that enhance tissue penetration, including, but not limited to zonulin, propylene glycol, mono-, di- or tri-glycerides etc.

Exemplary additive components can further include compounds that stabilize poorly water soluble pharmaceutical agents. Exemplary additive components providing such stabilization include biocompatible polymers, for example albumins. Additional additive components are described in U.S. Pat. No. 7,034,765 (De et al.), the disclosure of which is incorporated herein by reference. Stabilization of suspensions and emulsions can also be provided by compounds, for example, such as surfactants (e.g. F68).

Other additives include saccharides. Saccharides can include monosaccharides, disaccharides, trisaccharides, oligosaccharides, and polysaccharides. Polysaccharides can be linear or branched polysaccharides. Exemplary saccharides can include but are not limited to dextrose, sucrose, maltose, mannose, trehalose, and the like. Exemplary saccharides can further include, but are not limited to, polysaccharides including pentose, and/or hexose subunits, specifically including glucans such as glycogen and amylopectin, and dextrins including maltodextrins, fructose, mannose, galactose, and the like. Polysaccharides can also include gums such as pullulan, arabinose, galactan, etc.

Saccharides can also include derivatives of polysaccharides. It will be appreciated that polysaccharides include a variety of functional groups that can serve as attachment points or can otherwise be chemically modified in order to alter characteristics of the saccharide. As just one example, it will be appreciated that saccharide backbones generally include substantial numbers of hydroxyl groups that can be utilized to derivatize the saccharide. Saccharides can also include copolymers and/or terpolymers, and the like, that include saccharide and/or saccharide subunits and/or blocks.

Polysaccharides used with embodiments herein can have various molecular weights. By way of example, glycogen used with embodiments herein can have a molecular weight of greater than about 250,000. In some embodiments glycogen used with embodiments herein can have a molecular weight of between about 100,000 and 10,000,000 Daltons.

Refinement of the molecular weight of polysaccharides can be carried out using diafiltration. Diafiltration of polysaccharides such as maltodextrin can be carried out using ultrafiltration membranes with different pore sizes. As an example, use of one or more cassettes with molecular weight cut-off membranes in the range of about 1K to about 500 K can be used in a diafiltration process to provide polysaccharide preparations with average molecular weights in the range of less than 500 kDa, in the range of about 100 kDa to about 500 kDa, in the range of about 5 kDa to about 30 kDa, in the range of about 30 kDa to about 100 kDa, in the range of about 10 kDa to about 30 kDa, or in the range of about 1 kDa to about 10 kDa.

It will be appreciated that polysaccharides such as maltodextrin and amylose of various molecular weights are commercially available from a number of different sources. For example, Glucidex™ 6 (avg. molecular weight ˜95,000 Da) and Glucidex™ 2 (avg. molecular weight ˜300,000 Da) are available from Roquette (France); and MALTRIN™ maltodextrins of various molecular weights, including molecular weights from about 12,000 Da to 15,000 Da are available from GPC (Muscatine, Iowa). Examples of other hydrophobic polysaccharide derivatives are disclosed in US Patent Publication 2007/0260054 (Chudzik), which is incorporated herein by reference.

In another embodiment, the composition includes one or more amphiphilic additive. Amphiphilic compounds include those having a relatively hydrophobic portion and a relatively hydrophilic portion. Exemplary amphiphilic compounds can include, but are not limited to, polymers including, at least blocks of, polyvinylpyrrolidone, polyvinyl alcohol, polyethylene glycol, polyoxazolines (such as poly(2-alkyloxazoline) and derivatives) and the like. Exemplary amphiphilic compounds can specifically include poloxamers. Poloxamers are nonionic triblock copolymers composed of a central hydrophobic chain of polyoxypropylene flanked by two hydrophilic chains of polyoxyethylene. Poloxamers are frequently referred to by the trade name PLURONIC®. It will be appreciated that many aspects of the copolymer can be varied such the characteristics can be customized. One exemplary poloxamer is PLURONIC® F68 (non-ionic, co-polymer of ethylene and propylene oxide commercially available from BASF Corporation; also designated as F68 and poloxamer F68), which refers to a poloxamer having a solid form at room temperature, a polyoxypropylene molecular mass of approximately 1,800 g/mol and roughly 80% polyoxyethylene content, with a total molecular weight of approximately 8,400 g/mol, the copolymer terminating in primary hydroxyl groups.

In yet other embodiments, additive components can further include additives that effectively reverse the effect of drug uptake in tissue. Exemplary components that induce this reversal effect include heparin and heparin derivatives. Other negatively charged additive components that can complex with the cationic delivery agent of the present disclosure can also provide this reversal effect.

### Pharmaceutically Acceptable Carrier

In various embodiments, the active agent composition includes a hydrophobic active agent and a cationic delivery agent in a pharmaceutically acceptable carrier. In some embodiments, this can be an aqueous carrier. As used herein, a “pharmaceutically acceptable carrier” refers to a carrier or diluent that does not cause significant irritation to an organism and does not abrogate the biological activity and properties of the administered composition. In various embodiments, the aqueous carrier includes water or buffered saline. In a more particular embodiment, the aqueous carrier includes distilled water, double distilled water or distilled deionized water. In various embodiments, the hydrophobic active agent and/or the cationic delivery agent are suspended in water. In various embodiments, the carrier includes a minor amount (e.g., less than about 20%, 15%, 10%, 9%, 8%, 7%, 6%, 5%, 4%, 3%, 2% or 1%) of a biocompatible solvent. As used herein, the term “biocompatible solvent” refers to a solvent that is considered non-toxic and does not elicit an immunological response at the amounts included in the carrier. Examples of biocompatible solvents include, but are not limited to, ethanol, ethyl lactate, acetone, dimethylsulfoxide (DMSO), and combinations thereof. In various embodiments, the hydrophobic active agent is suspended in water as a coated therapeutic agent. In various embodiments, a mixing or agitation step can be performed in order to allow the hydrophobic active agent to interface with the cationic delivery agent. In some embodiments, the cationic delivery agent surrounds and/or encapsulates the particulate hydrophobic active agent to form a coated active agent particle.

In various embodiments, the pH of the composition is adjusted to at least about 5, 6 or 7 and up to about 7, 8 or 9.

### Method of Making

Some embodiments are directed towards methods of making the active agent compositions described herein. In some embodiments, the hydrophobic active agent can be processed, for example, by milling of the active agent. In some embodiments, processing of the hydrophobic active agent can include crystallization. In other embodiments, processing of the hydrophobic active agent can include lyophilizing of the active agent.

In various embodiments, the hydrophobic active agent is suspended in an aqueous carrier such as water. By combining the hydrophobic active agent and a cationic delivery agent, coated active agent particles can be formed. By way of example, a cationic agent, in water or other aqueous solvent, can be added to a hydrophobic active agent suspension. In some embodiments, a mixing or agitation step can be performed in order to allow the hydrophobic active agent to interface with the cationic agent. In some embodiments, the cationic agent will surround or encapsulate the particulate hydrophobic active agent. In various embodiments, the hydrophobic active agent has a particle size of at least about 0.1 μm, 0.2 μm, 0.3 μm, 0.4 μm, 0.5 μm or 1 μm and less than about 10 μm, 5 μm, 4 μm, 3 μm, 2 μm or 1 μm.

In various embodiments, an active agent solution or suspension is first made by combining a hydrophobic active agent with an aqueous solvent to form an active agent solution or suspension. After the active agent solution or suspension is formed, the cationic delivery agent is added to form an active agent composition. In various embodiments, the hydrophobic active agent is crystallized before it is combined with the aqueous solvent to form the active agent solution or suspension. In another embodiment, the hydrophobic active agent is amorphous when it is combined with the aqueous solvent to form the active agent solution or suspension. In another embodiment, the cationic delivery agent is combined with an aqueous solvent to form a cationic delivery agent solution before the cationic delivery agent is combined with the active agent solution or suspension. In various embodiments, the pH of the cationic delivery agent solution is buffered to between about 5 and 9 before the cationic delivery agent is added to the active agent solution or suspension.

In another embodiment, the active agent composition is made by combining the hydrophobic active agent and the cationic delivery agent to form an active agent mixture. In various embodiments, the active agent mixture comprises solid hydrophobic active agent and pure or neat cationic delivery agent. In various embodiments, the solid hydrophobic active agent is crystalline. In another embodiment, the solid hydrophobic active agent is amorphous. In various embodiments, the method includes a step of crystallizing the hydrophobic active agent before it is combined with the cationic delivery agent. In another embodiment, the hydrophobic active agent is amorphous when it is combined with the cationic delivery agent. In various embodiments, the method includes a step of crystallizing the mixture of solid hydrophobic active agent and pure or neat delivery agent before combining the mixture with an aqueous carrier to form the active agent composition. In another embodiment, a mixture containing crystalline hydrophobic active agent and pure or neat delivery agent is combined with the aqueous carrier to form the active agent composition. In general, when solid hydrophobic active agent and solid hydrophobic cationic delivery agent are combined to form a mixture, the ratio of solid hydrophobic active agent:cationic delivery agent is less than about 1:5 to prevent the cationic delivery agent from solubilizing the hydrophobic active agent.

### Kits and Articles of Manufacture

Another embodiment herein is directed towards kits and articles of manufacture. In particular, kits or packages including the active agent compositions described herein and/or catheters described herein are included. In an embodiment, a kit includes one or more of the components of the active agent composition. As used herein “components of the active agent composition” can refer to one or more hydrophobic active agents, one or more cationic delivery agents, one or more pharmaceutically acceptable carriers, and any other additive, diluent, excipient, adjuvant, emulsifier, buffer, stabilizer, preservative included in the active agent composition. In various embodiments, the kit includes one or more hydrophobic active agents and one or more cationic delivery agent and instructions for combining the hydrophobic active agent and cationic delivery agent to form an active agent composition suitable for local administration. In various embodiments, the cationic delivery agent includes PEI. In another embodiment, the cationic delivery agent includes branched PEI. In a specific embodiment, the hydrophobic active agent is paclitaxel, sirolimus (rapamycin), everolimus, biolimus A9, zotarolimus, tacrolimus, and pimecrolimus and mixtures thereof.

In various embodiments, the kit includes at least about 1 mg/ml and up to about 25 mg/ml cationic delivery agent and at least about 5 mg/ml and up to about 125 mg/ml hydrophobic active agent, wherein the components are packaged individually, or combined, for example as a mixture of solids or as a liquid solution or suspension. In various embodiments, the kit includes at least about 1 mg/ml, 2 mg/ml, 3 mg/ml, 4 mg/ml, 5 mg/ml, or 10 mg/ml, 15 mg/ml, 20 mg/ml, or 25 mg/ml or up to about 25 mg/ml, 50 mg/ml, 75 mg/ml, 100 mg/ml, 125 mg/ml or 150 mg/ml hydrophobic active agent. In various embodiments, the kit includes at least about 0.1 mg/ml, 0.5 mg/ml, 1 mg/ml, 2 mg/ml, 3 mg/ml, 4 mg/ml, or 5 mg/ml and up to about 5 mg/ml, 10 mg/ml, 15 mg/ml, 20 mg/ml or 25 mg/ml cationic delivery agent. In various embodiments, the kit includes cationic delivery agent:hydrophobic active agent at a ratio of at least 1:25, for example, between about 1:1 and about 1:25, or at least about 1:2, 1:5 or 1:10 and up to about 1:10, 1:15, 1:20 or 1:25.

The components of the active agent composition (for example, the hydrophobic active agent, the cationic delivery agent, the pharmaceutically acceptable aqueous carrier and/or any other additives) may be individually formulated or co-formulated and filled into suitable containers such as syringes, ampoules, or vials. It is envisioned that the carrier also may be provided in another container in the kit. The kits herein will typically include a means for containing the vials in close confinement for commercial sale such as, for example, injection or blow-molded plastic containers into which the desired vials are retained. In various embodiments, the kit also includes a catheter as described herein.

In various embodiments, the kit provides one or more of the components of the active agent composition and instructions for combining the components for administration. In various embodiments, one or more of the components of the active agent composition in the kit is provided in dried or lyophilized forms. In various embodiments, the hydrophobic active agent, the cationic delivery agent, or both are provided as dried solids, individually or as a mixture. In another embodiment, the hydrophobic active agent, the cationic delivery agent, or both are provided as lyophilized solids, individually or as a mixture. In various embodiments, the hydrophobic active agent, the cationic delivery agent, or both are provided as amorphous solids, individually or as a mixture. In another embodiment, the hydrophobic active agent, the cationic delivery agent, or both are provided as crystalline solids, individually or as a mixture. When one or more components are provided as a dried solid, reconstitution generally is by the addition of a suitable carrier. In various embodiments, the carrier is an aqueous carrier.

In another embodiment, one or more of the components of the active agent composition is provided as a solution or suspension. In various embodiments, the hydrophobic active agent, the cationic delivery agent, or both are provided as a solution or suspension, individually or as a mixture. For example, if individually provided, two solution components can be separated in a dual delivery syringe for ease of delivery to the site (for example dual delivery syringes and mini-dual delivery syringes available from Plas-Pak, Inc, Norwich, Conn.). In some instances, contents of a dual delivery syringe can be lyophilized to provide for a dual delivery syringe that contains a solution or suspension in one side and a dry powder in the other. Alternatively, the dual delivery syringe can contain lyophilized dry powder in both sides of the dual syringe. It is well known in the art that the lyophilized powder can be reconstituted at the point of use with physiologically acceptable fluid, such as phosphate buffered saline (PBS).

In various embodiments, one or more of the components of the active agent composition are provided as a dried solid in a container, individually or as a mixture, for example, as a crystallized solid or an amorphous solid, and are reconstituted with a pharmaceutically acceptable carrier prior to administration. In other embodiments, one or more of the components of the active agent composition are provided in as a liquid, in a container, individually or as a mixture, that may be administered with or without dilution. In various embodiments, one of the components of the active agent composition may be provided in solid form, which, prior to administration to a patient, is reconstituted with an aqueous liquid and another component of the active agent composition may be provided as a liquid solution or suspension, wherein the components are combined prior to administration. Each container may contain a unit dose of the active agent(s).

In some examples, portions of the access device 100, catheter 130, or both may be covered with a coating. For example, hydrophilic polymeric base coatings can be applied to portions of the medical device to impart lubricity and decrease particulate shedding. In some examples, portions of the valve on the distal end of the sleeve are covered with a coating. In other examples, the inner diameter of the sleeve is coated or lined with lubricious low friction coatings or the outer diameter is lined with lubricious low friction coatings, friction reducing or lubricating materials such as silicone oil, perfluorinated oils or waxes or with covalently bonded coating that imparts lower friction.

### Low Friction Surfaces

Exemplary embodiments of low friction surfaces for the vascular access devices described herein include substrates prepared from low friction materials (e.g. PTFE and PTFE liners) and surfaces that can be made to be low friction by addition of coatings (e.g. coatings with hydrophilic polymers).

One class of hydrophilic polymers useful as polymeric materials for hydrophilic base coat formation can be synthetic hydrophilic polymers. Synthetic hydrophilic polymers that are biostable (i.e., that show no appreciable degradation in vivo) can be prepared from any suitable monomer including acrylic monomers, vinyl monomers, ether monomers, or combinations of any one or more of these types of monomers. Acrylic monomers include, for example, methacrylate, methyl methacrylate, hydroxyethyl methacrylate, hydroxyethyl acrylate, methacrylic acid, acrylic acid, glycerol acrylate, glycerol methacrylate, acrylamide, methacrylamide, dimethylacrylamide (DMA), and derivatives and/or mixtures of any of these. Vinyl monomers include, for example, vinyl acetate, vinylpyrrolidone, vinyl alcohol, and derivatives of any of these. Ether monomers include, for example, ethylene oxide, propylene oxide, butylene oxide, and derivatives of any of these. Examples of polymers that can be formed from these monomers include poly(acrylamide), poly(methacrylamide), poly(vinylpyrrolidone), poly(acrylic acid), poly(ethylene glycol), poly(vinyl alcohol), and poly(HEMA). Examples of hydrophilic copolymers include, for example, methyl vinyl ether/maleic anhydride copolymers and vinyl pyrrolidone/(meth)acrylamide copolymers. Mixtures of homopolymers and/or copolymers can be used.

Examples of some acrylamide-based polymers, such as poly(N,Ndimethylacrylamide-co-aminopropylmethacrylamide) and poly(acrylamide-co-N,Ndimethylaminopropylmethacrylamide) are described in example 2 of U.S. Pat. No. 7,807,750 (Taton et al.), the disclosure of which is incorporated herein by reference.

Other hydrophilic polymers that can be useful in the present disclosure are derivatives of acrylamide polymers with photoreactive groups. One such representative hydrophilic polymer can be the copolymerization of N-[3-(4-benzoylbenzamido)propyl]methacrylamide (Formula I) with N-(3-aminopropyl)methacrylamide (Formula II) to produce the polymer poly(N-3-aminopropyl)methacrylamide-co-N-[3-(4-benzoylbenzamido)propyl]methacrylamide (Formula III). The preparation of the polymer is disclosed in Example 1 of US Patent Publication 2007/0032882 (to Lodhi, et al.), the full content of which is incorporated herein by reference.

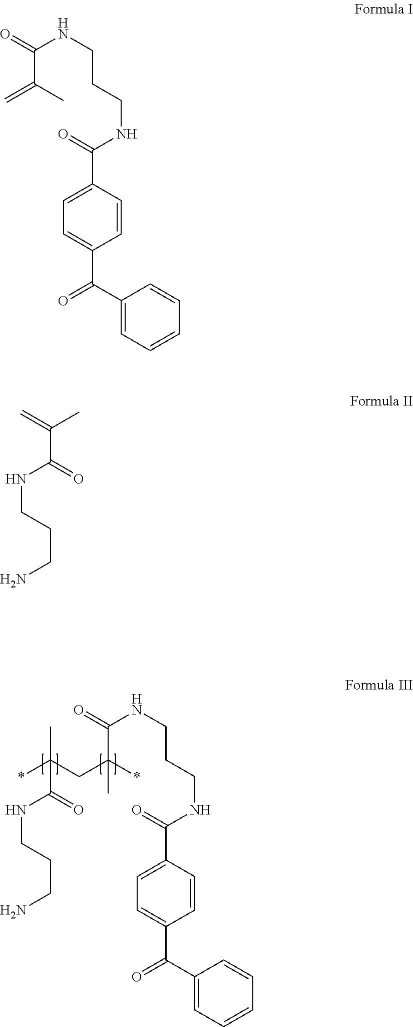

In some embodiments, the hydrophilic polymer can be a vinyl pyrrolidone polymer, or a vinyl pyrrolidone/(meth)acrylamide copolymer such as poly(vinylpyrrolidone-co-methacrylamide). If a PVP copolymer is used, it can be a copolymer of vinylpyrrolidone and a monomer selected from the group of acrylamide monomers. Exemplary acrylamide monomers include (meth)acrylamide and (meth)acrylamide derivatives, such as alkyl(meth)acrylamide, as exemplified by dimethylacrylamide, and aminoalkyl(meth)acrylamide, as exemplified by aminopropylmethacrylamide and dimethylaminopropylmethacrylamide. For example, poly(vinylpyrrolidone-co-N,N-dimethylaminopropylmethacrylamide) is described in example 2 of U.S. Pat. No. 7,807,750 (Taton et al.).

In one embodiment, the polymers and copolymers as described are derivatized with one or more photoactivatable group(s). Exemplary photoreactive groups that can be pendent from biostable hydrophilic polymer include aryl ketones, such as acetophenone, benzophenone, anthraquinone, anthrone, quinone, and anthrone-like heterocycles. Aryl ketones herein can specifically include diaryl ketones. Polymers herein can provide a hydrophilic polymer having a pendent activatable photogroup that can be applied to the expandable and collapsible structure, and can then treated with actinic radiation sufficient to activate the photogroups and cause covalent bonding to a target, such as the material of the expandable and collapsible structure. Use of photo-hydrophilic polymers can be used to provide a durable coating of a flexible hydrogel matrix, with the hydrophilic polymeric materials covalently bonded to the material of the expandable and collapsible structure.

A hydrophilic polymer having pendent photoreactive groups can be used to prepare the flexible hydrogel coating. Methods of preparing hydrophilic polymers having photoreactive groups are known in the art. For example, methods for the preparation of photo-PVP are described in U.S. Pat. No. 5,414,075, the disclosure of which is incorporated herein by reference. Hydrophilic photo-polyacrylamide polymers such as poly(acrylamide-co-N-(3-(4-benzoylbenzamido)propyl)methacrylamide), “Photo PA”, and derivatives thereof can be used to form hydrophilic base coats in exemplary embodiments of the present disclosure. Methods for the preparation of photo-polyacrylamide are described in U.S. Pat. No. 6,007,833, the disclosure of which is incorporated herein by reference.

Other embodiments of hydrophilic base coats include derivatives of photo-polyacrylamide polymers incorporating additional reactive moieties. Some exemplary reactive moieties include N-oxysuccinimide and glycidyl methacrylate. Representative photo-polyacrylamide derivatives incorporating additional reactive moieties include poly(acrylamide-co-maleic-6-aminocaproic acid-N-oxysuccinimide-co-N-(3-(4-benzoylbenzamido)propyl)methacrylamide) and poly(acrylamide-co-(3-(4-benzoylbenzamido)propyl)methacrylamide)-co-glycidylmethacrylate. Additional photo-polyacrylamide polymers incorporating reactive moieties are described in U.S. Pat. No. 6,465,178 (to Chappa, et al.), U.S. Pat. No. 6,762,019 (to Swan, et al.) and U.S. Pat. No. 7,309,593 (to Ofstead, et al.), the disclosures of which are herein incorporated by reference.

Other embodiments of exemplary hydrophilic base coats that include derivatives of photo-polyacrylamide polymers incorporating additional reactive moieties can be found in U.S. Pat. No. 6,514,734 (to Clapper, et al.), the disclosure of which is incorporated herein by reference in its entirety.

In yet other embodiments, the hydrophilic base coat can include derivatives of photo-polyacrylamide polymers incorporating charged moieties. Charged moieties include both positively and negatively charged species. Exemplary charged species include, but are not limited to, sulfonates, phosphates and quaternary amine derivatives. Some examples include the negatively charged species N-acetylated poly(acrylamide-co-sodium-2-acrylamido-2-methylpropanesulfonate-co-N-(3-(4-benzoylbenzamido)propyl)methacrylamide)-co-methoxy poly(ethylene glycol) monomethacrylate. Other negatively charged species that can be incorporated into the hydrophilic base coat are described in U.S. Pat. No. 4,973,993, the disclosure of which is incorporated herein by reference in its entirety. Positively charged species can include poly(acrylamide-co-N-(3-(4-benzoylbenzamido)propyl)methacrylamide)-co-(3-(methacryloylamino)propyl)trimethylammonium chloride. Other positively charged species that can be incorporated into the hydrophilic base coat are described in U.S. Pat. No. 5,858,653 (to Duran et al.), the disclosure of which is incorporated herein by reference in its entirety.

In another embodiment, the polymers and copolymers as described are derivatized with one or more polymerizable group(s). Polymers with pendent polymerizable groups are commonly referred to as macromers. The polymerizable group(s) can be present at the terminal portions (ends) of the polymeric strand or can be present along the length of the polymer. In one embodiment polymerizable groups are located randomly along the length of the polymer.

Exemplary hydrophilic polymer coatings can be prepared using polymer grafting techniques. Polymer grafting techniques can include applying a nonpolymeric grafting agent and monomers to a substrate surface then causing polymerization of the monomers on the substrate surface upon appropriate activation (for example, but not limited to, UV radiation) of the grafting agent. Grafting methods producing hydrophilic polymeric surfaces are exemplified in U.S. Pat. Nos. 7,348,055; 7,736,689 and 8,039,524 (all to Chappa et al.) the full disclosures of which are incorporated herein by reference.

Optionally, the coating can include a crosslinking agent. A crosslinking agent can promote the association of polymers in the coating, or the bonding of polymers to the coated surface. The choice of a particular crosslinking agent can depend on the ingredients of the coating composition.

Suitable crosslinking agents can include two or more activatable groups, which can react with the polymers in the composition. Suitable activatable groups can include photoreactive groups as described herein, like aryl ketones, such as acetophenone, benzophenone, anthraquinone, anthrone, quinone, and anthrone-like heterocycles. A crosslinking agent including a photoreactive group can be referred to as a photo-crosslinker or photoactivatable crosslinking agent. The photoactivatable crosslinking agent can be ionic, and can have good solubility in an aqueous composition. Thus, in some embodiments, at least one ionic photoactivatable crosslinking agent can be used to form the coating. The ionic crosslinking agent can include an acidic group or salt thereof, such as selected from sulfonic acids, carboxylic acids, phosphonic acids, salts thereof, and the like. Exemplary counter ions include alkali, alkaline earths metals, ammonium, protonated amines, and the like.

Exemplary ionic photoactivatable crosslinking agents include 4,5-bis(4-benzoylphenylmethyleneoxy) benzene-1,3-disulfonic acid or salt; 2,5-bis(4-benzoylphenylmethyleneoxy)benzene-1,4-disulfonic acid or salt; 2,5-bis(4-benzoylmethyleneoxy)benzene-1-sulfonic acid or salt; N,N-bis[2-(4-benzoylbenzyloxy)ethyl]-2-aminoethanesulfonic acid or salt, and the like. See U.S. Pat. No. 6,077,698 (Swan et al.), U.S. Pat. No. 6,278,018 (Swan), U.S. Pat. No. 6,603,040 (Swan) and U.S. Pat. No. 7,138,541 (Swan) the disclosures of which are incorporated herein by reference.

Other exemplary ionic photoactivatable crosslinking agents include ethylenebis(4-benzoylbenzyldimethylammonium) dibromide and hexamethylenebis(4-benzoylbenzyldimethylammonium) dibromide and the like. See U.S. Pat. No. 5,714,360 (Swan et al.) the disclosures of which are incorporated herein by reference.

In yet other embodiments, restrained multifunctional reagents with photoactivable crosslinking groups can be used. In some examples these restrained multifunctional reagents include tetrakis (4-benzoylbenzyl ether) of pentaerthyritol and the tetrakis (4-benzoylbenzoate ester) of pentaerthyritol. See U.S. Pat. No. 5,414,075 (Swan et al.) and U.S. Pat. No. 5,637,460 (Swan et al.) the disclosures of which are incorporated herein by reference.

Additional crosslinking agents can include those having formula Photo1-LG-Photo2, wherein Photo1 and Photo2 independently represent at least one photoreactive group and LG represents a linking group including at least one silicon or at least one phosphorus atom, wherein the degradable linking agent comprises a covalent linkage between at least one photoreactive group and the linking group, wherein the covalent linkage between at least one photoreactive group and the linking group is interrupted by at least one heteroatom. See U.S. Pat. No. 8,889,760 (Kurdyumov, et al.), the disclosure of which is incorporated herein by reference. Further crosslinking agents can include those having a core molecule with one or more charged groups and one or more photoreactive groups covalently attached to the core molecule by one or more degradable linkers. See U.S. Publ. Pat. App. No. 2011/0144373 (Swan, et al.), the disclosure of which is incorporated herein by reference.

Crosslinking agents used in accordance with embodiments herein can include those with at least two photoreactive groups. Exemplary crosslinking agents are described in U.S. Pat. No. 8,889,760, the content of which is herein incorporated by reference in its entirety.

In some embodiments, the first and/or second crosslinking agent can have a molecular weight of less than about 1500 kDa. In some embodiments the crosslinking agent can have a molecular weight of less than about 1200, 1100, 1000, 900, 800, 700, 600, 500, or 400.

In some embodiments, at least one of the first and second crosslinking agents including a linking agent having formula Photo1-LG-Photo2, wherein Photo1 and Photo2, independently represent at least one photoreactive group and LG represents a linking group including at least one silicon or at least one phosphorus atom, there is a covalent linkage between at least one photoreactive group and the linking group, wherein the covalent linkage between at least one photoreactive group and the linking group is interrupted by at least one heteroatom.

In some embodiments, at least one of the first and second crosslinking agents including a linking agent having a formula selected from:

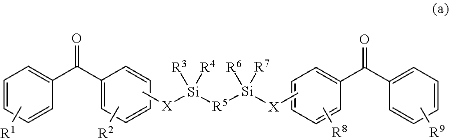

wherein R1, R2, R8 and R9 are any substitution; R3, R4, R6 and R7 are alkyl, aryl, or a combination thereof; R5 is any substitution; and each X, independently, is O, N, Se, S, or alkyl, or a combination thereof;

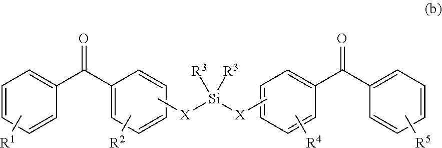

wherein R1 and R5 are any substitution; R2 and R4 can be any substitution, except OH; R3 can be alkyl, aryl, or a combination thereof; and X, independently, are 0, N, Se, S, alkylene, or a combination thereof;

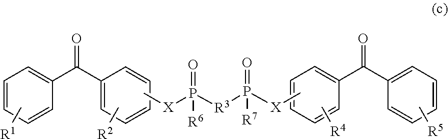

wherein R1, R2, R4 and R5 are any substitution; R3 is any substitution; R6 and R7 are alkyl, aryl, or a combination thereof; and each X can independently be O, N, Se, S, alkylene, or a combination thereof; and

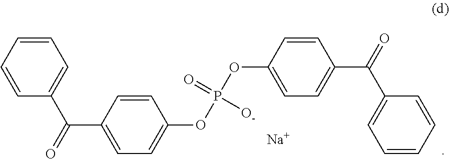

In a particular embodiment, the crosslinking agent can be bis(4-benzoylphenyl) phosphate.

In some embodiments, the photoactivatable crosslinking agent can be ionic, and can have good solubility in an aqueous composition, such as the first and/or second coating composition. Thus, in some embodiments, at least one ionic photoactivatable crosslinking agent is used to form the coating. In some cases, an ionic photoactivatable crosslinking agent can crosslink the polymers within the second coating layer which can also improve the durability of the coating.

Any suitable ionic photoactivatable crosslinking agent can be used. In some embodiments, the ionic photoactivatable crosslinking agent is a compound of formula I: X1-Y—X2 where Y is a radical containing at least one acidic group, basic group, or a salt of an acidic group or basic group. X1 and X2 are each independently a radical containing a latent photoreactive group. The photoreactive groups can be the same as those described herein. Spacers can also be part of X1 or X2 along with the latent photoreactive group. In some embodiments, the latent photoreactive group includes an aryl ketone or a quinone.

The radical Y in formula I provides the desired water solubility for the ionic photoactivatable crosslinking agent. The water solubility (at room temperature and optimal pH) is at least about 0.05 mg/ml. In some embodiments, the solubility is about 0.1 to about 10 mg/ml or about 1 to about 5 mg/ml.

In some embodiments of formula I, Y is a radical containing at least one acidic group or salt thereof. Such a photoactivatable crosslinking agent can be anionic depending upon the pH of the coating composition. Suitable acidic groups include, for example, sulfonic acids, carboxylic acids, phosphonic acids, and the like. Suitable salts of such groups include, for example, sulfonate, carboxylate, and phosphate salts. In some embodiments, the ionic crosslinking agent includes a sulfonic acid or sulfonate group. Suitable counter ions include alkali, alkaline earths metals, ammonium, protonated amines, and the like.

For example, a compound of formula I can have a radical Y that contains a sulfonic acid or sulfonate group; X1 and X2 can contain photoreactive groups such as aryl ketones. Such compounds include 4,5-bis(4-benzoylphenylmethyleneoxy)benzene-1,3-disulfonic acid or salt; 2,5-bis(4-benzoylphenylmethyleneoxy)benzene-1,4-disulfonic acid or salt; 2,5-bis(4-benzoylmethyleneoxy)benzene-1-sulfonic acid or salt; N,N-bis[2-(4-benzoylbenzyloxy)ethyl]-2-aminoethanesulfonic acid or salt, and the like. See U.S. Pat. No. 6,278,018. The counter ion of the salt can be, for example, ammonium or an alkali metal such as sodium, potassium, or lithium.

In other embodiments of formula I, Y can be a radical that contains a basic group or a salt thereof. Such Y radicals can include, for example, an ammonium, a phosphonium, or a sulfonium group. The group can be neutral or positively charged, depending upon the pH of the coating composition. In some embodiments, the radical Y includes an ammonium group. Suitable counter ions include, for example, carboxylates, halides, sulfate, and phosphate. For example, compounds of formula I can have a Y radical that contains an ammonium group; X1 and X2 can contain photoreactive groups that include aryl ketones. Such photoactivatable crosslinking agents include ethylenebis(4-benzoylbenzyldimethylammonium) salt; hexamethylenebis (4-benzoylbenzyldimethylammonium) salt; 1,4-bis(4-benzoylbenzyl)-1,4-dimethylpiperazinediium) salt, bis(4-benzoylbenzyl)hexamethylenetetraminediium salt, bis[2-(4-benzoylbenzyldimethylammonio)ethyl]-4-benzoylbenzylmethylammonium salt; 4,4-bis(4-benzoylbenzyl)morpholinium salt; ethylenebis[(2-(4-benzoylbenzyldimethylammonio)ethyl)-4-benzoylbenzylmethylammonium] salt; and 1,1,4,4-tetrakis(4-benzoylbenzyl)piperzinediium salt. See U.S. Pat. No. 5,714,360. The counter ion is typically a carboxylate ion or a halide. On one embodiment, the halide is bromide.

In other embodiments, the ionic photoactivatable crosslinking agent can be a compound having the formula:

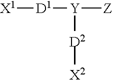

wherein X1 includes a first photoreactive group; X2 includes a second photoreactive group; Y includes a core molecule; Z includes at least one charged group; D1 includes a first degradable linker; and D2 includes a second degradable linker. Additional exemplary degradable ionic photoactivatable crosslinking agents are described in US Patent Application Publication US 2011/0144373 (Swan et al., “Water Soluble Degradable Crosslinker”), the disclosure of which is incorporated herein by reference.

In some aspects a non-ionic photoactivatable crosslinking agent can be used. In one embodiment, the non-ionic photoactivatable crosslinking agent has the formula XR1R2R3R4, where X is a chemical backbone, and R1, R2, R3, and R4 are radicals that include a latent photoreactive group. Exemplary non-ionic crosslinking agents are described, for example, in U.S. Pat. Nos. 5,414,075 and 5,637,460 (Swan et al., “Restrained Multifunctional Reagent for Surface Modification”). Chemically, the first and second photoreactive groups, and respective spacers, can be the same or different.

In other embodiments, the non-ionic photoactivatable crosslinking agent can be represented by the formula:

PG2-LE2-X-LE1-PG1

wherein PG1 and PG2 include, independently, one or more photoreactive groups, for example, an aryl ketone photoreactive group, including, but not limited to, aryl ketones such as acetophenone, benzophenone, anthraquinone, anthrone, anthrone-like heterocycles, their substituted derivatives or a combination thereof; LE1 and LE2 are, independently, linking elements, including, for example, segments that include urea, carbamate, or a combination thereof; and X represents a core molecule, which can be either polymeric or non-polymeric, including, but not limited to a hydrocarbon, including a hydrocarbon that is linear, branched, cyclic, or a combination thereof; aromatic, non-aromatic, or a combination thereof; monocyclic, polycyclic, carbocyclic, heterocyclic, or a combination thereof; benzene or a derivative thereof; or a combination thereof. Other non-ionic crosslinking agents are described, for example, in U.S. application Ser. No. 13/316,030 filed Dec. 9, 2011 (Publ. No. US 2012/0149934) (Kurdyumov, “Photocrosslinker”), the disclosure of which is incorporated herein by reference.

Further embodiments of non-ionic photoactivatable crosslinking agents can include, for example, those described in US Pat. Publication 2013/0143056 (Swan et al., “Photo-Vinyl Primers/Crosslinkers”), the disclosure of which is incorporated herein by reference. Exemplary crosslinking agents can include non-ionic photoactivatable crosslinking agents having the general formula R1-X—R2, wherein R1 is a radical including a vinyl group, X is a radical including from about one to about twenty carbon atoms, and R2 is a radical including a photoreactive group.

A single photoactivatable crosslinking agent or any combination of photoactivatable crosslinking agents can be used in forming the coating. In some embodiments, at least one nonionic crosslinking agent such as tetrakis(4-benzoylbenzyl ether) of pentaerythritol can be used with at least one ionic crosslinking agent. For example, at least one non-ionic photoactivatable crosslinking agent can be used with at least one cationic photoactivatable crosslinking agent such as an ethylenebis(4-benzoylbenzyldimethylammonium) salt or at least one anionic photoactivatable crosslinking agent such as 4,5-bis(4-benzoyl-phenylmethyleneoxy)benzene-1,3-disulfonic acid or salt. In another example, at least one nonionic crosslinking agent can be used with at least one cationic crosslinking agent and at least one anionic crosslinking agent. In yet another example, a least one cationic crosslinking agent can be used with at least one anionic crosslinking agent but without a non-ionic crosslinking agent.

An exemplary crosslinking agent is disodium 4,5-bis[(4-benzoylbenzyl)oxy]-1,3-benzenedisulfonate (DBDS). This reagent can be prepared by combining 4,5-Dihydroxylbenzyl-1,3-disulfonate (CHBDS) with 4-bromomethylbenzophenone (BMBP) in THF and sodium hydroxide, then refluxing and cooling the mixture followed by purification and recrystallization (also as described in U.S. Pat. No. 5,714,360, incorporated herein by reference).

Further crosslinking agents can include the crosslinking agents described in U.S. Publ. Pat. App. No. 2010/0274012 (to Guire et al.) and U.S. Pat. No. 7,772,393 (to Guire et al.) the content of all of which is herein incorporated by reference.

In some embodiments, crosslinking agents can include boron-containing linking agents including, but not limited to, the boron-containing linking agents disclosed in US Pat. Publication 2013/0302529 entitled “Boron-Containing Linking Agents” by Kurdyumov et al., the content of which is herein incorporated by reference. By way of example, linking agents can include borate, borazine, or boronate groups and coatings and devices that incorporate such linking agents, along with related methods. In an embodiment, the linking agent includes a compound having the structure (I):

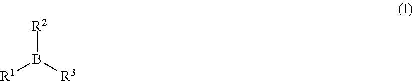

wherein R1 is a radical including a photoreactive group; R2 is selected from OH and a radical including a photoreactive group, an alkyl group and an aryl group; and R3 is selected from OH and a radical including a photoreactive group. In some embodiments the bonds B—R1, B—R2 and B—R3 can be chosen independently to be interrupted by a heteroatom, such as O, N, S, or mixtures thereof.

Additional agents for use with embodiments herein can include stilbene-based reactive compounds including, but not limited to, those disclosed in U.S. Pat. No. 8,487,137, entitled “Stilbene-Based Reactive Compounds, Polymeric Matrices Formed Therefrom, and Articles Visualizable by Fluorescence” by Kurdyumov et al., the content of which is herein incorporated by reference.

Additional photoreactive agents, crosslinking agents, hydrophilic coatings, and associated reagents are disclosed in U.S. Pat. No. 8,513,320 (to Rooijmans et al.); U.S. Pat. No. 8,809,411 (to Rooijmans); and 2010/0198168 (to Rooijmans), the content of all of which is herein incorporated by reference.

Natural polymers can also be used to form the hydrophilic base coat. Natural polymers include polysaccharides, for example, polydextrans, carboxymethylcellulose, and hydroxymethylcellulose; glycosaminoglycans, for example, hyaluronic acid; polypeptides, for example, soluble proteins such as collagen, albumin, and avidin; and combinations of these natural polymers. Combinations of natural and synthetic polymers can also be used.

In some instances a tie layer can be used to form the hydrophilic base layer. In yet other instances the tie layer can be added to the hydrophilic base layer. The tie layer can act to increase the adhesion of the hydrophilic base layer to the substrate. In other embodiments, the tie layer can act to increase adhesion of the hydrophobic active agent to the hydrophilic base layer. Exemplary ties layers include, but are not limited to silane, butadiene, polyurethane and parylene. Silane tie layers are described in US Patent Publication 2012/0148852 (to Jelle, et al.), the content of which is herein incorporated by reference.

In exemplary embodiments, the hydrophilic base layer can include tannic acid, polydopamine or other catechol containing materials.

The above detailed description is intended to be illustrative, and not restrictive. The scope of the disclosure should, therefore, be determined with references to the appended claims, along with the full scope of equivalents to which such claims are entitled.

As used herein, the term “organ” refers to a functional grouping of one or more tissues. Functionally related organs may cooperate to form organ systems. Examples of organs and organ systems found in mammals include, but are not limited to: the cardiovascular system, which includes organs such as the heart and blood vessels; the digestive system, which includes organs such as salivary glands, esophagus, stomach, liver, tongue, gallbladder, pancreas, intestines, colon, rectum and anus; the endocrine system, which includes endocrine glands such as the hypothalamus, pituitary gland, pineal body or pineal gland, thyroid, parathyroid and adrenal glands; the excretory system, which includes organs such as kidneys, ureters, bladder and urethra; the immune system, which includes tonsils, adenoids, thymus and spleen; the integumentary system, which includes skin, hair and nails; the muscular system, which includes voluntary and involuntary muscles; the nervous system, which includes brain, spinal cord and nerves; the reproductive system, which includes the sex organs, such as ovaries, fallopian tubes, uterus, vagina, mammary glands, testes, vas deferens, seminal vesicles, prostate and penis; the respiratory system, which includes the pharynx, larynx, trachea, bronchi, lungs and diaphragm; and the skeletal system, which includes bones, cartilage, ligaments and tendons. As used herein, the terms “tissue” and “organs” refer to solid tissues or organs, rather than blood or other biological liquids such as spinal fluid, amniotic fluid or peritoneal fluid.

As used herein, an “individual” or a “patient” is a vertebrate, for example, a mammal. The term “mammal” can also refer to any animal classified as a mammal, including humans, domestic and farm animals, and zoo, sports, or pet animals, such as dogs, horses, cats, cows, etc. In a more particular embodiment, the mammal is human.

The term “effective amount” refers to an amount effective, at dosages and for periods of time necessary, to achieve the desired therapeutic or prophylactic result. A “therapeutically effective amount” of the active agent composition may vary according to factors such as the disease state, age, sex, and weight of the individual, and the ability of the substance/molecule, agonist or antagonist to elicit a desired response in the individual. A therapeutically effective amount is also one in which any toxic or detrimental effects of the composition are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount may be less than the therapeutically effective amount.

In various embodiments, a method for treating a tissue or organ of a patient is included. As used herein, the terms “treat”, “treating” and “treatment” refer to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishing of any direct or indirect pathological consequences of the disease, decreasing the rate of disease progression, amelioration or palliation of the disease state, and remission or improved prognosis. As used herein, the terms “prevent”, “preventing” and “prevention” refer to a method for preventing an organism from acquiring a disorder.

### Further Embodiments

In an embodiment, a method of treating a localized region of the body with an active agent composition is included. The method can include inserting a catheter into the lumen of a blood vessel of a patient, the catheter including a shaft including a proximal end and a distal end, the catheter further including an inflatable balloon, a first lumen disposed within the shaft for delivering a fluid to inflate the balloon, an active agent delivery port, and a second lumen disposed within the shaft for delivering the active agent composition through the shaft to the active agent delivery port. The method can also include inflating the balloon to at least partially occlude the flow of blood through the blood vessel of the patient. The method can also include ejecting the active agent composition out of the active agent delivery port into the blood vessel. The active agent composition can include a cationic delivery agent, a therapeutically effective amount of the hydrophobic active agent, and a pharmaceutically acceptable carrier. The method can also include deflating the balloon to restore the flow of blood through the blood vessel; and withdrawing the catheter from the lumen of the blood vessel of the patient.

In some embodiments, inflating the balloon completely occludes the flow of blood in the vessel. In some embodiments, inflating the balloon causes the peak systolic velocity of blood flow through the artery to be reduced by more than 75%. In some embodiments, inflating the balloon causes the peak systolic velocity of blood flow through the artery to be reduced to less than 2 cm/sec.

In some embodiments, the blood vessel comprises a vein. In some embodiments, the blood vessel comprises an artery. In some embodiments, the blood vessel comprises a renal artery. In some embodiments, the blood vessel comprises a hepatic artery.

In some embodiments, the method can include moving the catheter within the lumen of the blood vessel to position the balloon in a desired location prior the step of inflating the balloon.

In some embodiments, the catheter includes a tip on the distal end of the catheter, the active agent delivery port disposed within 3 centimeters of the tip. In some embodiments, the active agent delivery port is positioned to eject the active agent composition is a direction parallel to the lengthwise axis of the catheter. In some embodiments, the active agent delivery port is positioned to eject the active agent composition is a direction perpendicular to the lengthwise axis of the catheter.

In some embodiments, at least 30 seconds lapses between when the step of ejecting the active agent composition begins and the step of deflating the balloon begins. In some embodiments, the catheter further includes a guidewire shaft.

In some embodiments, the cationic delivery agent is selected from the group consisting of cationic lipids, net neutral lipids with a cationic group, and cationic polymers. In some embodiments, the cationic delivery agent selected from the group consisting of polyethylenimine (PEI), 1,2-dioleoyl-3-trimethylammonium-propane (DOTAP), polyamidoamine dendrimers (PAMAM). polyvinylamine (PVAm). In some embodiments, the cationic delivery agent comprises branched PEI.

In some embodiments, the active agent composition includes cationic delivery agent:hydrophobic active agent at a ratio of at least about 1:1 and up to about 1:25. In some embodiments, the active agent composition including a cationic delivery agent:hydrophobic active agent at a ratio of at least about 1:2 and up to about 1:20. In some embodiments, the active agent composition including cationic delivery agent:hydrophobic active agent at a ratio of at least about 1:5 and up to about 1:10.

In some embodiments, the active agent composition including at least about 1 mg/ml and up to about 25 mg/ml cationic delivery agent. In some embodiments, the active agent composition including at least about 5 mg/ml and up to about 125 mg/ml hydrophobic active agent. In some embodiments, the aqueous carrier is selected from water and buffered saline.

In some embodiments, the active agent composition having a pH between 5 and 9. In some embodiments, the active agent composition having a pH between 6 and 8. In some embodiments, the active agent composition having a pH between 7 and 8.

In some embodiments, the hydrophobic active agent is selected from an antiproliferative, analgesic, anti-inflammatory, anti-arrhythmic, anti-bacterial, anti-coagulant, anti-hypertensive, anti-muscarinic, anti-neoplastic, beta-blocker, cardiac inotropic agent, corticosteroids, lipid regulating agents, anti-anginal agents, and combinations thereof. In some embodiments, the hydrophobic active agent comprises an antiproliferative selected from paclitaxel, sirolimus, everolimus, biolimus A9, zotarolimus, tacrolimus, and pimecrolimus and mixtures thereof.

In some embodiments, the pharmaceutically acceptable carrier is an aqueous carrier.

In some embodiments, a catheter for treating a localized region of the body with an active agent composition is included. The catheter can include a shaft including a proximal end and a distal end, an inflatable balloon, a first lumen disposed within the shaft for delivering a gas to inflate the balloon, an active agent delivery port, and a second lumen disposed within the shaft in fluid communication with the active agent delivery port. An active agent composition can be disposed within the second lumen. The active agent composition can include a cationic delivery agent, a therapeutically effective amount of the hydrophobic active agent and a pharmaceutically acceptable carrier.

In some embodiments, a kit for treating a localized region of the body with an active agent composition is included. The kit can include a catheter including a shaft including a proximal end and a distal end, an inflatable balloon, a first lumen disposed within the shaft for delivering a gas to inflate the balloon, an active agent delivery port, and a second lumen disposed within the shaft in fluid communication with the active agent delivery port. The kit can also include an active agent composition disposed within the second channel. The active agent composition can include a cationic delivery agent, a therapeutically effective amount of the hydrophobic active agent and a pharmaceutically acceptable carrier.

In some embodiments, a method of treating a localized region of the body with an active agent composition is included. The method can include inserting a catheter into the lumen of a blood vessel of a patient, the catheter including a shaft including a proximal end and a distal end, the catheter further including an inflatable balloon, a first lumen disposed within the shaft for delivering a fluid to inflate the balloon, an active agent delivery port, and a second lumen disposed within the shaft for delivering the active agent composition through the shaft to the active agent delivery port. The method can also include inflating the balloon to at least partially occlude the flow of blood through the blood vessel of the patient. The method can also include ejecting the active agent composition out of the active agent delivery port into the blood vessel. The active agent composition can include a bioactive agent-containing particulate. The particulate can include a core particle including bioactive agent and a first biocompatible polymer and a layer around the core particle including a second biocompatible polymer and that comprises negatively charged groups, wherein the second biocompatible polymer is more water soluble than the first biocompatible polymer. Instead, or in addition, the particle can include a bioactive agent and a first biocompatible polymer that is water insoluble, wherein the polymer is chemically modified at the particle surface to provide negatively charged groups; and a cationic agent associated with the negatively charged groups of the particle, deflating the balloon to restore the flow of blood through the blood vessel and withdrawing the catheter from the lumen of the blood vessel of the patient.

### EXAMPLES

### Example 1: Transcatheter Drug Delivery In-Vivo

Suspensions were prepared as prepared
(A) Paclitaxel @ 1.2 mg/mL
(B) Paclitaxel/PEI (92:8 by wt) @ 1.2 mg/mL
(C) Paclitaxel/pVam (92:8 by wt) @ 1.2 mg/mL
(D) Paclitaxel/cremophor with paclitaxel at 6.0 mg/mL; diluted 5× with saline (1.2 mg/mL final concentration)

### Occlusion Devices

VENOS® Occlusion Balloon Catheter (6Fr and 7 Fr devices; available from Oscor; Palm Harbor, Fla.).

Targeted Organs

| No | Organ | Vessel |
|---|---|---|
| 1 | Spleen | Gastro splenic distal artery |
| 2 | Stomach-1 | Gastro splenic proximal main artery |
| 3 | Stomach-2 | Gastro splenic proximal branched artery |
| 4 | Liver | Gastro hepatic cranial lobe artery |
| 5 | Pancreas | Gastro hepatic caudal artery |
| 6 | Tongue | Lingual artery |
| 7 | Kidney-1 | Left renal artery |
| 8 | Kidney-2 | Right renal artery |

### Procedures

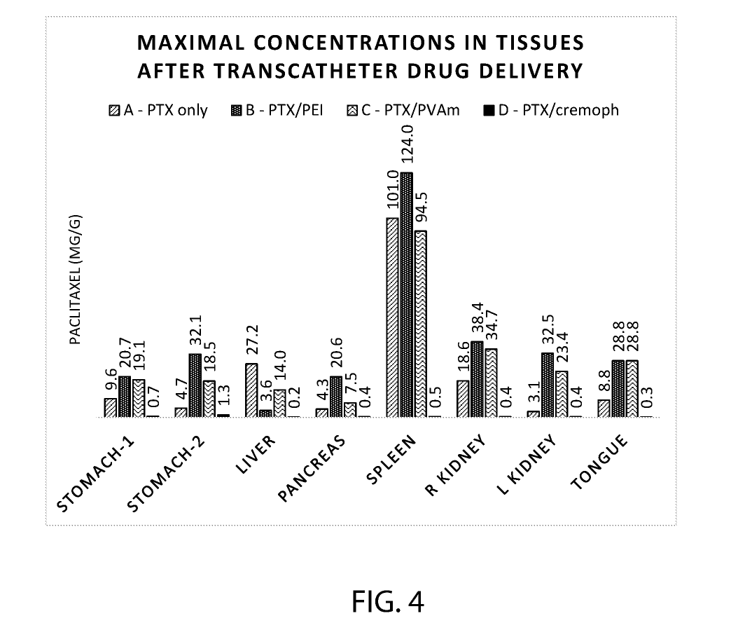

Using a VENOS® Occlusion Balloon Catheter arterial feeders of target organs described above were occluded with the balloon. Contrast media was delivered via a Y-connector with a Touhy Borst valve connected to the guide wire lumen of the catheter and the volume needed to reach the organ was recorded. Four mL of the above suspensions (A-D) were delivered, followed by the same amount of contrast media as delivered before the drug formulation to confirm drug administration to the targeted organ. Organs were harvested and analyzed for paclitaxel concentration. The results are shown in FIG. 4.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration to. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this disclosure pertains. All publications and patent applications are herein incorporated by reference to the same extent as if each individual publication or patent application was specifically and individually indicated by reference.

Aspects have been described with reference to various specific and preferred exemplary embodiments and techniques. However, it should be understood that many variations and modifications may be made while remaining within the spirit and scope herein.

1. A method of treating a localized region of the body with an active agent composition comprising:
inserting a catheter into the lumen of a blood vessel of a patient, the catheter comprising a shaft including a proximal end and a distal end, the catheter further comprising an inflatable balloon, a first lumen disposed within the shaft for delivering a fluid to inflate the balloon, an active agent delivery port, and a second lumen disposed within the shaft for delivering the active agent composition through the shaft to the active agent delivery port;
inflating the balloon to at least partially occlude the flow of blood through the blood vessel of the patient;
ejecting the active agent composition out of the active agent delivery port into the blood vessel, the active agent composition comprising:
a cationic delivery agent;
a therapeutically effective amount of the hydrophobic active agent; and
a pharmaceutically acceptable carrier;
deflating the balloon to restore the flow of blood through the blood vessel; and
withdrawing the catheter from the lumen of the blood vessel of the patient.

2. The method of claim 1, wherein inflating the balloon completely occludes the flow of blood in the vessel.

3. The method of claim 1, wherein inflating the balloon causes the peak systolic velocity of blood flow through the artery to be reduced by more than 75%.

4. The method of claim 1, wherein inflating the balloon causes the peak systolic velocity of blood flow through the artery to be reduced to less than 2 cm/sec.

5. The method of claim 1, further comprising moving the catheter within the lumen of the blood vessel to position the balloon in a desired location prior the step of inflating the balloon.

6. The method of claim 1, the catheter comprising a tip on the distal end of the catheter, the active agent delivery port disposed within 3 centimeters of the tip.

7. The method of claim 1, wherein at least 30 seconds lapses between when the step of ejecting the active agent composition begins and the step of deflating the balloon begins.

8. The method of claim 1, wherein the cationic delivery agent is selected from the group consisting of cationic lipids, net neutral lipids with a cationic group, and cationic polymers.

9. The method of claim 1, the cationic delivery agent selected from the group consisting of polyethylenimine (PEI), 1,2-dioleoyl-3-trimethylammonium-propane (DOTAP), polyamidoamine dendrimers (PAMAM). polyvinylamine (PVAm).

10. The method of claim 1, wherein the cationic delivery agent comprises branched PEI.

11. The method of claim 1, the active agent composition comprising cationic delivery agent:hydrophobic active agent at a ratio of at least about 1:1 and up to about 1:25.

12. The method of claim 1, the active agent composition comprising cationic delivery agent:hydrophobic active agent at a ratio of at least about 1:2 and up to about 1:20.

13. The method of claim 1, the active agent composition comprising at least about 1 mg/ml and up to about 25 mg/ml cationic delivery agent.

14. The method of claim 1, the active agent composition comprising at least about 5 mg/ml and up to about 125 mg/ml hydrophobic active agent.

15. The method of claim 1, the active agent composition having a pH between 6 and 8.

16. The method of claim 1, the active agent composition having a pH between 7 and 8.

17. The method of claim 1, wherein the hydrophobic active agent is selected from an antiproliferative, analgesic, anti-inflammatory, anti-arrhythmic, anti-bacterial, anti-coagulant, anti-hypertensive, anti-muscarinic, anti-neoplastic, beta-blocker, cardiac inotropic agent, corticosteroids, lipid regulating agents, anti-anginal agents, and combinations thereof.

18. The method of claim 1, wherein the hydrophobic active agent comprises an antiproliferative selected from paclitaxel, sirolimus, everolimus, biolimus A9, zotarolimus, tacrolimus, and pimecrolimus and mixtures thereof.

19. A catheter for treating a localized region of the body with an active agent composition comprising:
a shaft including a proximal end and a distal end,
an inflatable balloon,
a first lumen disposed within the shaft for delivering a gas to inflate the balloon,
an active agent delivery port, and
a second lumen disposed within the shaft in fluid communication with the active agent delivery port;
an active agent composition disposed within the second lumen, the active agent composition comprising:
a cationic delivery agent;
a therapeutically effective amount of the hydrophobic active agent; and
a pharmaceutically acceptable carrier.

20. A kit for treating a localized region of the body with an active agent composition comprising:
a catheter comprising:
a shaft including a proximal end and a distal end,
an inflatable balloon,
a first lumen disposed within the shaft for delivering a gas to inflate the balloon,
an active agent delivery port, and
a second lumen disposed within the shaft in fluid communication with the active agent delivery port; and
an active agent composition disposed within the second channel, the active agent composition comprising:
a cationic delivery agent;
a therapeutically effective amount of the hydrophobic active agent; and
a pharmaceutically acceptable carrier.
